(19) United States
(12) Patent Application Publication
(10) Pub. No.: US 2013/0084594 A1
(43) Pub. Date: Apr. 4, 2013

(54) DRUG SUSCEPTIBILITY USING RARE CELL DETECTION SYSTEM

(71) Applicant: Battelle Memorial Institute, Columbus, OH (US)

(72) Inventors: Herbert S. Bresler, Bexley, OH (US); Kevin M. Taylor, Columbus, OH (US); John S. Laudo, Hilliard, OH (US); Thomas D. Haubert, Columbus, OH (US)

(73) Assignee: BATTELLE MEMORIAL INSTITUTE, Columbus, OH (US)

(21) Appl. No.: 13/622,645

(22) Filed: Sep. 19, 2012

Related U.S. Application Data
(63) Continuation-in-part of application No. 13/225,074, filed on Sep. 2, 2011.
Application No. 13/225,074 is a continuation of application No. 13/052,151, filed on Mar. 21, 2011, now Pat. No. 8,012,742.
Application No. 13/052,151 is a continuation of application No. 12/029,274, filed on Feb. 11, 2008, now Pat. No. 7,915,029.
Application No. 12/029,274 is a continuation of application No. 11/370,635, filed on Mar. 7, 2006, now Pat. No. 7,329,534.
Application No. 11/370,635 is a division of application No. 10/263,975, filed on Oct. 3, 2002, now Pat. No. 7,074,577.
Application No. 10/263,975 is a continuation-in-part of application No. 12/498,533, filed on Jul. 7, 2009.
Application No. 12/498,533 is a division of application No. 11/519,533, filed on Sep. 12, 2006, now Pat. No. 7,560,277.
Application No. 11/519,533 is a division of application No. 11/261,105, filed on Oct. 27, 2005, now Pat. No. 7,397,601.
Application No. 11/261,105 is a continuation-in-part of application No. 13/371,761, filed on Feb. 13, 2012.
Application No. 13/371,761 is a continuation-in-part of application No. 13/052,172, filed on Mar. 21, 2011, now Pat. No. 8,114,680.
Application No. 13/052,172 is a continuation of application No. 12/614,602, filed on Nov. 9, 2009, now Pat. No. 7,919,049.
Application No. 12/614,602 is a continuation of application No. 12/029,289, filed on Feb. 11, 2008, now Pat. No. 7,629,176.
Application No. 12/029,289 is a continuation of application No. 11/609,186, filed on Dec. 11, 2006, now Pat. No. 7,358,095.
Application No. 11/609,186 is a continuation of application No. 10/263,974, filed on Oct. 3, 2002, now Pat. No. 7,220,593.
(60) Provisional application No. 61/536,303, filed on Sep. 19, 2011.
(60) Provisional application No. 60/631,025, filed on Nov. 24, 2004.
(60) Provisional application No. 60/631,026, filed on Nov. 24, 2004.
(60) Provisional application No. 60/631,027, filed on Nov. 24, 2004.

(51) Int. Cl. C12Q 1/02

(52) U.S. Cl. CPC C12Q 1/025
USPC 435/29

(57) ABSTRACT

Methods for determining the efficacy of a given drug for a specific patient with cancer in vitro prior to, or after, the initiation of treatment of the patient are disclosed. Blood from the cancer patient is separated into an assay test tube and a control test tube. The blood in the assay test tube is exposed to a cancer drug. The two test tubes are then visually examined and compared to determine the effect of the cancer drug on cancer cells, other rare cells in the blood, or on normal constituents of the blood of a cancer patient.

### CROSS-REFERENCE TO RELATED APPLICATIONS

This application claims priority to U.S. Provisional Patent Application Ser. No. 61/536,303, filed on Sep. 19, 2011. This application is also a continuation-in-part of U.S. patent application Ser. No. 13/225,074, filed Sep. 2, 2011, which is a continuation of U.S. Pat. No. 8,012,742, filed Mar. 21, 2011, which was a continuation of U.S. Pat. No. 7,915,029, filed Feb. 11, 2008, which was a continuation of U.S. Pat. No. 7,329,534, filed Mar. 7, 2006, which was a divisional of U.S. Pat. No. 7,074,577, filed Oct. 3, 2002. This application is also a continuation-in-part of U.S. patent application Ser. No. 12/498,533, filed Jul. 7, 2009, which is a divisional of U.S. Pat. No. 7,560,277, filed Sep. 12, 2006, which was a divisional application of U.S. Pat. No. 7,397,601, filed Oct. 27, 2005, which claimed priority to three different provisional applications: U.S. Provisional Patent Application Ser. No. 60/631,025, filed Nov. 24, 2004; U.S. Provisional Patent Application Ser. No. 60/631,026, filed Nov. 24, 2004; and U.S. Provisional Patent Application Ser. No. 60/631,027, filed Nov. 24, 2004. This application is also a continuation-in-part of U.S. patent application Ser. No. 13/371,761, filed Feb. 13, 2012, which is a continuation of U.S. Pat. No. 8,114,680, filed on Mar. 21, 2011, which was a continuation of U.S. Pat. No. 7,919,049, filed Nov. 9, 2009, which was a continuation of U.S. Pat. No. 7,629,176, filed Feb. 11, 2008, which was a continuation of U.S. Pat. No. 7,358,095, filed Dec. 11, 2006, which was a continuation of U.S. Pat. No. 7,220,593, filed Oct. 3, 2002. The disclosures of these applications are hereby incorporated by reference in their entirety.

### BACKGROUND

The present disclosure relates to methods for determining the efficacy of a given drug for a specific patient with cancer in vitro prior to the initiation of treatment of the patient or after treatment to determine whether drug resistance has developed. The methods may also be useful in evaluating the toxicity of a given drug, or for quantifying the needed amount of a given drug. Generally, the methods may facilitate personalized medicine, enabling the choice and/or amount of drug to be tailored to the individual patient. The methods may also be useful experimentally as part of the drug development process.

Cancers, or malignant neoplasms, belong to a class of diseases in which a group of cells display uncontrolled growth, invade and destroy adjacent tissues, and metastasize (i.e. spread to other locations in the body). Cancers are one of the leading causes of disease in the world. They attack many different organs in the human body.

Cancers can be treated in many ways. Some drugs can be applied or taken by the cancer patient. Radiation treatment can be used to kill the cancerous tumor cells. Surgery can also be used to remove cancerous tumors, or the organs / body parts infected by such tumors.

In particular, many different types of anti-cancer drugs exist. These types include alkylating agents, antimetabolites, plant alkaloids, inhibitors of various enzymes, and monoclonal antibodies. Many different anti-cancer drugs have been synthesized. These drugs include, for example, vinblastine, vincristine, vinflunine, vindesine, vinorelbine, cabazitaxel, docetaxel, larotaxel, ortataxel, paclitaxel, tesetaxel, ixabepilone, aminopterin, methotrexate, pemetrexed, pralatrexate, raltitrexed, pemetrexed, pentostatin, cladribine, clofarabine, fludarabine, thioguanine, mercaptopurine, fluorouracil, capecitabine, tegafur, carmofur, floxuridine, cytarabine, gemcitabine, azacitidine, decitabine, hydroxycarbamide, camptothecin, topotecan, irinotecan, rubitecan, belotecan, etoposide, teniposide, aclarubicin, daunorubicin, doxorubicin, epirubicin, idarubicin, amrubicin, pirarubicin, valrubicin, zorubicin mitoxantrone, pixantrone, mechlorethamine, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, thiotepa, triaziquone, triethylenemelamine, carboplatin, cisplatin, nedaplatin, oxaliplatin, triplatin tetranitrate, satraplatin, procarbazine, dacarbazine, temozolomide, altretamine, mitobronitol, actinomycin, bleomycin, mitomycin, plicamycin, aminolevulinic acid/methyl aminolevulinate, efaproxiral, porfimer sodium, talaporfin, temoporfin, verteporfin, tipifamib, alvocidib, seliciclib, bortezomib, anagrelide, tiazofurine, masoprocol, olaparib, vorinostat, romidepsin, atrasentan, bexarotene, testolactone, amsacrine, trabectedin, alitretinoin, tretinoin, arsenic trioxide, asparaginase/pegaspargase, celecoxib, demecolcine, elesclomol, elsamitrucin, etoglucid, lonidamine, lucanthone, mitoguazone, mitotane, oblimersen, omacetaxine mepesuccinate, everolimus, temsirolimus, cetuximab, panitumumab, trastuzumab, catumaxomab, edrecolomab, bevacizumab, ibritumomab, ofatumumab, rituximab, tositumomab, alemtuzumab, gemtuzumab, erlotinib, gefitinib, vandetanib, afatinib, lapatinib, neratinib, axitinib, pazopanib, sunitinib, sorafenib, toceranib, lestaurtinib, axitinib, cediranib, pazopanib, regorafenib, semaxanib, sorafenib, sunitinib, toceranib, vandetanib, dasatinib, imatinib, nilotinib, bosutinib, lestaurtinib, crizotinib, aflibercept, and denileukin diftitox.

Unfortunately, anti-cancer drugs have many adverse side effects. These adverse side effects include immune system depression, infections, fatigue, tendency to bleed easily, nausea, vomiting, diarrhea, constipation, hair loss, damage to certain organs, infertility, pain or paralysis, and impotence. Many of these anti-cancer drugs target biological processes that simply occur in tumor cells at a faster rate than in healthy cells, but healthy cells can be affected as well. Each drug has a different side effect profile, and each drug also works differently between patients. In other words, a drug that works in one patient may be ineffective in another patient.

The conventional way of selecting an anti-cancer drug for use in a particular patient involves prescribing a drug and correcting the prescription based on an observation of the patient's response to the drug. This approach can be time-consuming and cause adverse side effects in the patient. It would be desirable to provide alternative methods or processes for determining what drugs are effective in a particular patient, or put another way which drugs the tumors in a particular patient are susceptible to.

### BRIEF DESCRIPTION

The present disclosure relates to methods and processes for testing drug susceptibility in a cancer patient, or for evaluating the toxicity of a drug, or for quantifying the appropriate dose of a drug for a patient. Briefly, blood from the cancer patient is divided into a control test tube and an assay test tube. The blood in the assay test tube is exposed to a particular drug. The assay test tube and the control test tube are then compared to each other to determine the effect of the drug on cancer cells or other rare cells in the patient's blood. This provides information to a physician on whether the particular drug may be beneficial to the cancer patient or may continue to benefit the patient.

In some embodiments, a method of testing for drug susceptibility in a cancer patient comprises dividing a blood sample of the cancer patient into a control test tube and an assay test tube. A drug is added to the assay test tube. A separator float is introduced into the assay test tube and moved into alignment with the cancer cells to capture the cancer cells in an annular volume. The assay test tube is then visually examined. The effect of the drug on cancer cells in the assay test tube is compared to cancer cells in the control test tube.

The change in the shape of the cancer cells, or the number of intact cancer cells, may be compared between the assay test tube and the control test tube.

The method may further comprise staining the cancer cells prior to visually examining the assay test tube. In addition, the control test tube may be visually examined. For example, the visual examination may detect the quantity of fluorescence in the assay test tube due to the staining (e.g. immunofluorescence).

The visual examination can be performed by introducing a separator float into the assay test tube. The assay test tube is then centrifuged to move the float into alignment with the cancer cells. Subsequently, the rotational speed is reduced or stopped) to capture the cancer cells in a volume between the test tube and the separator float. The cancer cells can then be examined. The visual examination can also be performed using an optical system that generates light having a non-uniform spatial distribution.

In particular embodiments, the separator float comprises a main body portion, a plurality of axially oriented ridges protruding from the main body portion, and does not have end sealing ridges.

In other embodiments, a method of testing for drug susceptibility in a cancer patient, comprises dividing a blood sample from the cancer patient into a control test tube and an assay test tube. A drug is added to the assay test tube. The assay test tube is visually examined to determine the effect of the drug on cancer cells in the blood. The cancer cells in the assay test tube are then compared with the cancer cells in the control test tube.

Another method of testing for drug susceptibility in a cancer patient comprises receiving a first test tube and a second test tube, each tube containing the blood of the cancer patient. A drug is added to the first test tube to make an assay test tube. The assay test tube is visually examined. The effect of the drug on cancer cells in the assay test tube is compared with cancer cells in the control test tube.

Still another method of testing for drug susceptibility in a cancer patient, comprises receiving a blood sample of the cancer patient and dividing the blood sample into a control test tube and an assay test tube. The blood in the assay test tube is mixed with a drug. The assay test tube is visually examined to determine the effect of the drug on a cell type in the blood. The effect on the cell type in the assay test tube is compared with the cell type in the control test tube.

Yet another method of testing for drug susceptibility in a cancer patient comprises dividing a blood sample of the cancer patient into a control test tube and an assay test tube. The blood in the assay test tube is exposed to a drug. The assay test tube is visually examined to determine the effect of the drug on a cell type in the blood. The effect on the cell type in the assay test tube is compared with the cell type in the control test tube.

Another method of testing for drug susceptibility in a cancer patient comprises dividing a blood sample of the cancer patient into a control test tube and a series of assay test tubes. The blood in the assay test tubes is exposed to a drug, with the quantity of the drug varying between assay test tubes. The assay test tubes are visually examined to determine the effect of the amount of the drug on a cell type in the blood. This can help determine the effective dose of the drug, in addition to whether or not the drug is effective.

These and other non-limiting aspects and/or objects of the disclosure are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a side view of a test tube and a separator float that can be used to visualize rare cells in a blood sample.

FIG. 2 is a diagram of a microscope system that can be used to visualize rare cells in a blood sample.

FIG. 3 is a perspective view of a test tube holder that can be used to with the system of FIG. 2, showing the internal components.

FIG. 4 is a side view of the test tube holder of FIG. 3, showing the internal components.

FIG. 5 is a perspective view of the test tube holder of FIG. 3, showing certain bearings.

FIG. 6 is a top view of the test tube holder of FIG. 3, showing certain internal components.

FIG. 7 is an embodiment of a separator float with end sealing ridges and radial support members.

FIG. 8 is an embodiment of a separator float with radial support members having a rectangular cross-section and no end sealing ridges.

FIG. 9 is an embodiment of a separator float with end sealing ridges and a helical support member having a rectangular cross-section.

FIG. 10 is an embodiment of a separator float with radial support members having a curved cross-section and no end sealing ridges.

FIG. 11 is an embodiment of a separator float with end sealing ridges and a helical support member having a curved cross-section.

FIG. 12 is an embodiment of a separator float with end sealing ridges and axially aligned support members.

FIG. 13 is a cross-sectional view of the float of FIG. 12.

FIG. 14 is an embodiment of a separator float with axially aligned support members and no end sealing ridges.

FIG. 15 is an embodiment of a separator float with end sealing ridges and axially aligned support members.

FIG. 16 is a perspective view of the float of FIG. 15.

FIG. 17 is an embodiment of a separator float with axially aligned support members and no end sealing ridges.

FIG. 18 is an embodiment of a separator float with end sealing ridges, radial ribs, and axially aligned splines.

FIG. 19 is an embodiment of a separator float with end sealing ridges and protrusions as support members, with the protrusions in a staggered pattern.

FIG. 20 is an embodiment of a separator float with protrusions in a staggered pattern and no end sealing ridges.

FIG. 21 is an embodiment of a separator float with end sealing ridges and protrusions as support members, with the protrusions in an aligned pattern.

FIG. 22 is an embodiment of a separator float with protrusions in an aligned pattern and no end sealing ridges.

FIG. 23 is a flowchart of one exemplary embodiment of the methods of the present disclosure.

FIG. 24 is an embodiment of a separator float having conical ends.

FIG. 25 is an embodiment of a separator float having frustoconical ends.

FIG. 26 is an embodiment of a separator float having convex or dome-shaped ends.

FIG. 27 is an embodiment of a separator float having the end sealing ridges offset from the ends of the main body portion.

FIG. 28 is an embodiment of a separator float having faceted protrusions and end sealing ridges.

FIG. 29 is an embodiment of a separator float having faceted protrusions and no end sealing ridges.

FIG. 30 is an embodiment of a separator float having a central bore and conical ends.

FIG. 31 is an embodiment of a separator float having a central bore, conical ends, and radially extending ribs.

FIG. 32 is an embodiment of a separator float having a central bore, conical ends, and axially extending ribs.

FIG. 33 is an exploded perspective view of a two-piece separator float.

FIG. 34 is a cross-sectional view of a two-piece separator float wherein the piston has a flanged end.

FIG. 35 is a cross-sectional view of a two-piece separator float having a flanged end and including tapered ends.

FIG. 36 is a cross-sectional view of a two-piece separator float which includes a central bore and a counterbore having different diameters.

FIG. 37 is a cross-sectional view of a two-piece separator float with a central bore and a counterbore having different diameters, and also having tapered ends.

FIG. 38 is a cross-sectional view of a two-piece separator float having a profiled bore and an enlarged head that interact.

FIG. 39 is a cross-sectional view of a two-piece separator float having a tapered internal passage.

FIG. 40 is a cross-sectional view of a two-piece separator float having an annular seat for the piston.

FIG. 41 is a diagram of a microscope system similar to that of FIG. 2, but with a modified optical system.

FIG. 42 is a diagram of a microscope system similar to that of FIG. 2, but with another modified optical system.

FIG. 43 is a diagram of a microscope system similar to that of FIG. 2, but with yet another modified optical system.

FIG. 44 is a top view of another test tube holder like FIG. 3, but using a test tube with an eccentric cross-section.

FIG. 45 is a top view of another test tube holder like FIG. 3, but using a test tube with an eccentric cross-section.

FIG. 46 is a side view of a portion of a test tube holder employing tilted roller bearings.

FIG. 47 is a side view of a portion of a test tube holder employing tilted roller bearings staggered along a test tube axis, along with a float having helical ridges enables spiral scanning of the test tube.

FIG. 48 is a perspective view of a test tube holder that holds the test tube horizontally and uses the test tube as a bias force.

FIG. 49 is a top view of a test tube holder employing bushing surfaces as alignment bearings and a set of ball bearings as bias bearings.

FIG. 50 diagrammatically depicts certain measurement parameters relevant in performing quantitative buffy coat analysis using a buffy coat sample trapped in an annular gap between an inside test tube wall and an outer surface of a float.

FIG. 51 diagrammatically shows a suitable quantitative buffy coat measurement/analysis approach.

FIG. 52 diagrammatically shows another suitable quantitative buffy coat measurement/analysis approach.

FIG. 53 diagrammatically shows a suitable image processing approach for tagging candidate cells.

FIG. 54 shows a pixel layout for a square filter kernel suitable for use in the matched filtering.

FIG. 55 shows a pixel intensity section A-A of the square filter kernel of FIG. 54.

FIG. 56 diagrammatically shows a suitable user verification process for enabling a human analyst to confirm or reject candidate cells.

### DETAILED DESCRIPTION

A more complete understanding of the processes and apparatuses disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

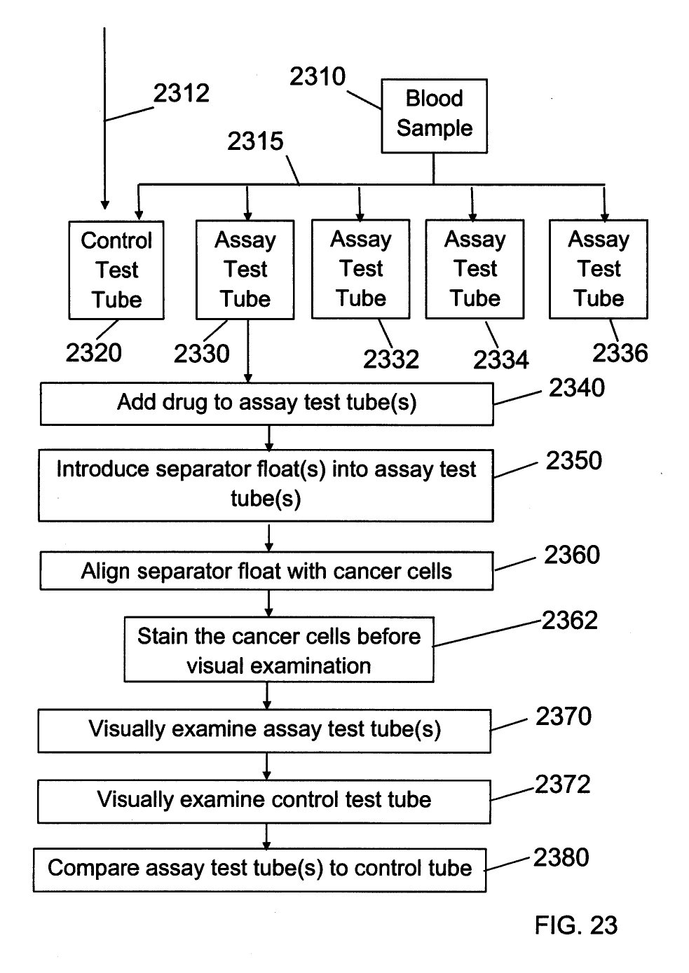

The present disclosure relates to a test for drug susceptibility using at least two test tubes and a rare cell detection system. The test is used to help determine whether a given drug will be useful for treating a cancer patient, and perhaps determining how much drug will be useful, before the given drug is actually administered to the patient. The drug is administered to at least one test tube (“the assay test tube”) and the other test tube is used as a “control test tube”. A series of assay test tubes which vary in the dose or concentration of the drug can also be used. The assay test tube(s) are then visually examined to determine the impact on the circulating cancer cells or other cells in the blood sample. This provides insight into the potential efficacy of the administered drug, and can also indicate an effective concentration or dose, and could also be used during drug research to identify appropriate drug candidates and their potential effective dose(s). FIG. 23 is a flowchart illustrating the test and its various steps. It should be noted that this is only one order in which the steps can be performed, and other orders of these steps are contemplated, as may be described further herein.

Initially, a control test tube and at least one assay test tube are obtained, each test tube containing blood from the cancer patient. Different methods for preparing the two test tubes are contemplated. For example, a blood sample of the cancer patient could be received for testing 2310. The blood sample can be procured from the cancer patient using normal procedures. The blood sample can be received in the form of one large sample that is subsequently divided 2315 into at least two smaller samples, corresponding to the control test tube 2320 and one or more assay test tubes 2330. Alternatively, the blood sample can be received in the form of two or more test tubes (i.e. at least a first test tube and a second test tube), which can be considered as having been divided prior to receipt, and as corresponding to the control test tube and the assay test tube (marked with reference numeral 2312).

Next, a drug 2340 is added to the assay test tube(s). Depending on the methods of the present disclosure, the drug may be one that is already known to have anti-cancer activity, or is a candidate being tested for anti-cancer activity. The assay test tube(s) can be shaken, mixed, or otherwise manipulated to ensure that the drug contacts or reacts with the various types of cells in the blood. In this regard, whole blood contains many different types of cells, including packed red cells, reticulocytes, granulocytes, lymphocytes/monocytes, platelets, and plasma, which can be separated by density. The blood of a cancer patient can also include circulating tumor cells and other rare cells of interest, such as certain epithelial cells which are associated with a specific type of cancer.

The assay test tube(s) are then visually examined 2370. The control test tube is also visually examined 2372, so that the results of the test tubes can be compared to each other 2380. The order in which the test tubes are examined is not important. This visual examination allows the effect of the drug on cancer cells in the blood to be determined. The effects provide information on the potential efficacy (and effective concentration) of the drug. The visual examination can be directed, for example, to the morphology of a given cell type in the blood sample or changes in the shape of the given cell. Alternatively, the visual examination might be for lysis of a given cell type, or put another way the quantity of intact cancer cells. As another example, the blood sample can be stained 2362 with an immunofluorescent agent that identifies specific cells or cell types, and the visual examination is then conducted to locate and examine those cells or cell types. The term “visual” refers to an examination of the test tubes using information gathered by light, rather than other means such as radioactivity or electrical patterns. For example, visual examination can be carried out using the human eye or a microscope to inspect the test tube and the cells contained within.

In this regard, immunofluorescence is a technique that uses the specificity of antibodies to their antigen to atttach fluorescent dyes to specific biomolecule targets within a cell, and therefore allows visualisation of the target molecules in the sample. Primary, or direct, immunofluorescence uses a single antibody that is chemically linked to a fluorophore. The antibody recognizes the target molecule and binds to it, and the fluorophore it carries can be detected via microscope. Secondary, or indirect, immunofluorescence uses two antibodies. The first or primary antibody recognises the target molecule and binds to it. The second or secondary antibody carries the fluorophore and binds to the primary antibody. Alternatively, the drug can be a fluorescently labeled drug, whose presence in the cell could then be visualized. Methods of fluorescent labeling a drug and such labeled drugs are known in the art. The quantity of fluorescence could be detected and/or measured using visual examination. The fluorescence can be measured in bulk or locally (e.g. between layers).

In this regard, it is known, for example, that a drug having an amine functional group can be reacted with dansyl chloride to produce stable fluorescent sulfonamide adducts. Known fluorophores include rhodamine, coumarin, fluorescein, and cyanine, and derivatives thereof. These fluorophores can be modified to label a drug. For example, a fluorescent dye iodoacetamide can be used to label a drug having a thiol functional group.

It is particularly contemplated that the visual examination of the assay test tube can be enhanced by using a separator float system. Briefly, this includes introducing a separator float 2350 into the assay test tube(s), and moving the separator float into alignment with the cancer cells 2360 in the test tube to capture those cancer cells in an annular volume, usually by centrifugal spinning of the test tube(s). This makes it easier to visualize the cancer cells. See FIG. 23.

It should be noted that the staining to enhance the visual examination (e.g. with a immunofluorescent agent) can be performed prior to introducing the separator float, or can be performed after the spinning of the test tube(s). For example, in FIG. 23, step 2362 may occur before step 2350 if desired. Similarly, the addition of the drug to the assay test tube(s) (step 2340) can be performed before or after introducing the separator float (step 2350), though the drug is usually added before the spinning of the tube/alignment of the separator float,

Specific embodiments are contemplated wherein more than one assay test tube is used. Different quantities (amount or concentration) of the drug can be added between different assay test tubes. This would permit a quantitative determination of the effective dose (if any) of the drug, rather than just a qualitative determination (works or does not work). As an example, four assay test tubes 2330, 2332, 2334, 2336 could be used, containing 1 mg/ml, 2 mg/ml, 4 mg/ml, and 8 mg/ml of the drug, respectively (see FIG. 23).

The testing conditions may vary, depending on the drug being tested. For example, the temperature of the test tubes can vary between room temperature (23-25° C.) to 37° C. The time for which the assay test tube(s) is exposed to the drug before visual examination may vary from minutes to hours. The pH of the liquid in the test tubes is likely to be maintained close to physiological (pH=7.4), but could vary from pH 7.1 to 7.6, or more ideally from pH 7.2 to 7.55.

Some specific systems are contemplated for use in visual examination of the two test tubes used in the methods of the present disclosure. One system is a buffy coat separator float system as described in U.S. patent application Ser. No. 10/263,974, the entirety of which is hereby incorporated by reference herein.

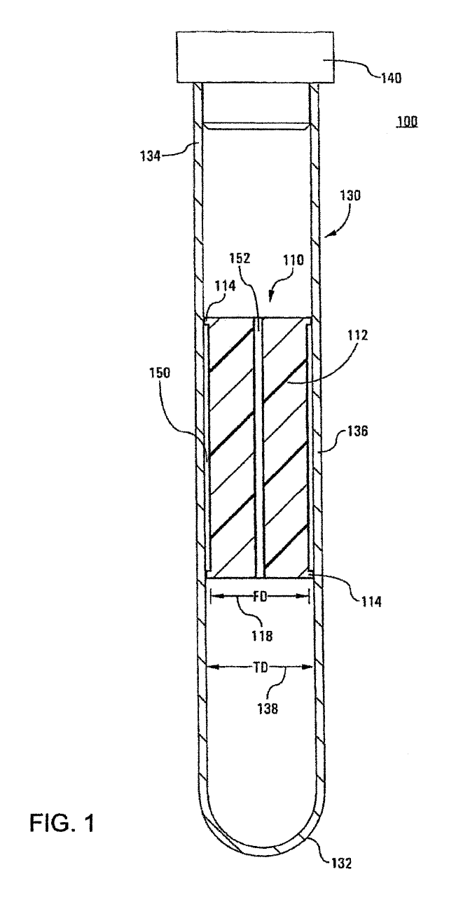

FIG. 1 shows a blood separation tube and float assembly 100, including a test tube 130 having a separator float 110, which can be used in the methods of the present disclosure. The test tube 130 is generally cylindrical. However, the tube 130 may be minimally tapered, slightly enlarging toward the open end 134, particularly when manufactured by an injection molding process. This taper or draft angle is generally desirable for ease of removal of the tube from the injection-molding tool. The test tube 130 includes a first, closed end 132 and a second open end 134 receiving a stopper or cap 140. Other closure means are also contemplated, such as parafilm or the like.

The tube 130 is formed of a transparent or semi-transparent material sufficient for visual examination. The sidewall 136 of the tube 130 is sufficiently flexible or deformable such that it expands in the radial direction during centrifugation, e.g., due to the resultant hydrostatic pressure of the sample under centrifugal load. As the centrifugal force is removed, the tube sidewall 136 substantially returns to its original size and shape.

The tube may be formed of any transparent or semi-transparent, flexible material. Preferably, the tube material is transparent. However, the tube does not necessarily have to be clear, as long as the receiving instrument that is looking for the cells or items of interest in the sample specimen can “see” or detect those items in the tube.

Preferably, the tube 130 is sized to accommodate the float 110 plus at least about five milliliters of blood or sample fluid, more preferably at least about eight milliliters of blood or fluid, and most preferably at least about ten milliliters of blood or fluid. In an especially preferred embodiment, the tube 130 has an inner diameter 138 of about 1.5 cm and accommodates a volume of from about two milliliters to about ten milliliters of blood in addition to the float 110.

The float 110 includes a main body portion 112 and one or more support members 114. In the embodiment shown here, the support members are seen as two sealing rings or flanges disposed at opposite axial ends (i.e. first and second ends, or top and bottom ends) of the float 110. The float 110 is formed of one or more generally rigid organic or inorganic materials, preferably a rigid plastic material. In this regard, the use of materials and/or additives that interfere with the visual examination method should be avoided. For example, if fluorescence is used, the material utilized to construct the float 110 should not have much “background” fluorescence at the wavelength of interest.

The main body portion 112 has an outer diameter 118 which is less than the inner diameter 138 of the test tube 130, under pressure or centrifugation. The support members 114 of the float generally have a diameter corresponding to the inner diameter 138 of the test tube 130. The main body portion 112 of the float, the support members 114, and the sidewall 136 of the tube 130 thereby define an annular channel or gap 150. The main body portion 112 occupies much of the cross-sectional area of the tube, the annular gap 150 being large enough to contain a specified portion of the blood sample. Preferably, the dimensions 118 and 138 are such that the annular gap 150 has a radial thickness ranging from about 25-250 microns, most preferably about 50 microns.

An optional bore or channel 152 may extend axially through the float 110. In this regard, the tube/float system can be centrifuged to separate the blood components by density. During centrifugation, the tube expands, freeing the float in the blood sample. As centrifugation is slowed, the float is captured by the wall 136 of the tube as it returns to its original diameter. As the tube continues to contract, pressure may build up in the blood fraction trapped below the float, primarily red blood cells. This pressure may cause cells to be forced into the annular channel 150 containing the captured blood components, thus making imaging of the contents of the annular channel more difficult. The bore 152 allows for any excessive fluid flow or any resultant pressure in the dense fractions trapped below the float 110 to be relieved. The excessive fluid flows into the bore 152, thus preventing degradation of the captured blood components. The bore extends completely from one end of the float to the other. In the preferred embodiment, the bore 152 is centrally located and extends axially.

As previously stated, the support members 114 are sized to be roughly equal to, or slightly greater than, the inner diameter 138 of the tube. The float 110, being generally rigid, can also provide support to the flexible tube wall 136. The seal formed between the support members 114 of the float and the wall 136 of the tube may be, but is not necessarily, a fluid-tight seal. As used herein, the term “seal” is also intended to encompass near-zero clearance or slight interference between the flanges 114 and the tube wall 136 providing a substantial seal, which is, in most cases, adequate for purposes of the disclosure.

In particular embodiments, the overall specific gravity of the separator float 110 should be between that of red blood cells (approximately 1.090) and that of plasma (approximately 1.028). In a preferred embodiment, the specific gravity is in the range of from about 1.089-1.029, more preferably from about 1.070 to about 1.040, and most preferably about 1.05. The overall specific gravity of the float 110 and the volume of the annular gap 150 may be selected so that the annular channel contains the buffy coat layers. The expanded buffy coat region can then be examined, e.g., under illumination and magnification, to identify circulating epithelial cancer or tumor cells or other target analytes.

In one preferred embodiment, the density of the float 110 is selected to ride in the granulocyte layer of the blood sample. The granulocytes ride in, or just above, the packed red-cell layer and have a specific gravity of about 1.08-1.09. In this preferred embodiment, the specific gravity of the float is in this range of from about 1.08 to about 1.09 such that, upon centrifugation, the float rides in the granulocyte layer. The amount of granulocytes can vary from patient to patient by as much as a factor of about twenty. Therefore, selecting the float density such that the float rides in the granulocyte layer is especially advantageous since loss of any of the lymphocyte/monocyte layer, which rides just above the granulocyte layer, is avoided. During centrifugation, as the granulocyte layer increases in size, the float rides higher in the granulocytes and keeps the lymphocytes and monocytes at essentially the same position with respect to the float. Generally the cells of greatest interest are the “mononuclear cells,” which includes principally monocytes and lymphocytes, as well as other cells of interest, cancer cells and other epithelial cells. The “buffy coat” layer includes all the white cells, including all of the granulocytes, the platelets, and the other leukocytes that are not mononuclear cells.

A fluorescently labeled antibody, which is specific to the target epithelial cells or other analytes of interest, can be added to the blood sample in the assay test tube and incubated. In an exemplary embodiment, the epithelial cells are labeled with anti-EpCAM having a fluorescent tag attached to it. Anti-EpCAM binds to an epithelial cell-specific site that is not expected to be present in any other cell normally found in the blood stream. A stain or colorant, such as acridine orange, may also be added to the sample to cause the various cell types to assume differential coloration for ease of discerning the buffy coat layers under illumination and to highlight or clarify the morphology of epithelial cells during examination of the sample. Alternatively, as previously described above, the drug could be fluorescently labeled itself, so that the location of the drug could be determined during visual examination.

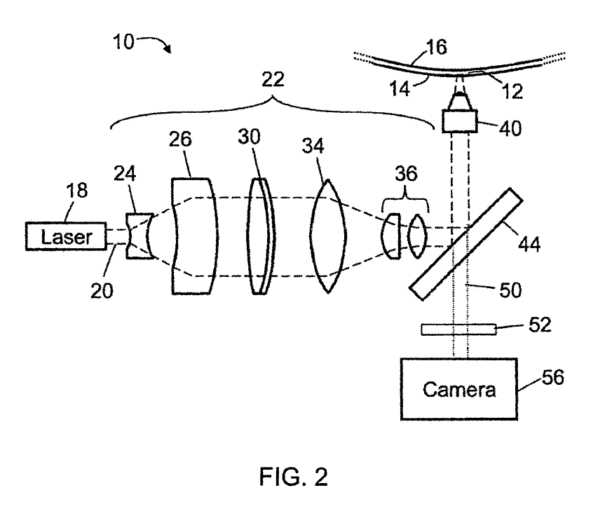

The separator float/tube system of FIG. 1 can be used to isolate the cancer cells or other cells of interest in the assay test tube and the control test tube. FIG. 2 shows a diagnostic system which can be used to visually examine the test tubes and determine the effect of the drug on one or more given cell types in the blood sample. This diagnostic system is described in more detail in U.S. Pat. No. 7,397,601, the entirety of which is hereby incorporated by reference herein.

Referring to FIG. 2, a microscope system 10 images a microscope field of view coinciding with a buffy coat sample disposed in a generally planar portion of an annular gap 12 between a light-transmissive test tube wall 14 and a float wall 16 of a float disposed in the test tube. The microscope field of view is generally planar in spite of the curvatures of the test tube and the float, because the microscope field of view is typically much smaller in size than the radii of curvature of the test tube wall 14 and the float wall 16. Although the field of view is substantially planar, the buffy coat sample disposed between the light-transmissive test tube wall 14 and the float wall 16 may have a thickness that is substantially greater than the depth of view of the microscope system 10. The test tube is mounted in fixed position respective to the microscope system 10 in a manner conducive to scanning the microscope field of view across the annular gap. As will be discussed, suitable mechanisms are preferably provided for effectuating relative rotational and/or translational scanning of the field of view over the annular gap containing the buffy coat sample.

The microscope system 10 may include a laser 18, such as a gas laser, a solid state laser, a semiconductor laser diode, or so forth, that generates source light 20 (indicated in FIG. 2 by dashed lines) in the form of a laser beam having an illumination wavelength and a non-uniform spatial distribution that is typically Gaussian or approximately Gaussian in shape with a highest intensity in a central region of the beam and reduced intensity with increasing distance from the beam center. An optical train 22 is configured to receive the spatially non-uniform source light 20 and to output a corrected spatial distribution.

A beam spreader includes a concave lens 24 that generally diverges the laser beam, and a collimating lens 26 that collimates the spread beam at a larger diameter that substantially matches the diameter of a Gaussian spatial characteristic of a beam homogenizer 30. The beam homogenizer 30 flattens the expanded laser beam by substantially homogenizing the Gaussian or other non-uniform distribution of the source light to produce output light having improved spatial uniformity. Alternatively, a stationary diffuser can be used as component 30. The diffuser may, for example, be a holographic diffuser. Such holographic diffusers employ a hologram providing randomizing non-periodic optical structures that diffuse the light to impart improved spatial uniformity. However, the diffusion of the light also imparts some concomitant beam divergence. Typically, stronger diffusion of the light tend to impart more spatial uniformity, but also tends to produce greater beam divergence. Holographic diffusers are suitably classified according to the full-width-at-half-maximum (FWHM) of the divergence angle, with larger divergence angles typically providing more diffusion and greater light uniformity, but also leading to increased light loss in the microscope system due to increased beam divergence.

A focusing lens 34 and cooperating lenses 36 reduce the expanded and flattened or homogenized laser beam down to a desired beam diameter for input to an objective 40 that is focused on the microscope field of view. A dichroic mirror 44 is selected to substantially reflect light at the wavelength or wavelength range of the laser beam, and to substantially transmit light at the fluorescence wavelength or wavelength range of the fluorescent dye used to tag rare cells in the buffy coat sample.

The optical train 22 including the stationary optical components 24, 26, 30, 34, 36 is configured to output a corrected spatial distribution to the objective 40 that when focused by the objective 40 at the microscope field of view provides substantially uniform static illumination over substantially the entire microscope field of view. The objective 40 focuses the corrected illumination onto the microscope field of view. The objective 40 may include a single objective lens, or may include two or more objective lenses. The focus depth of the microscope system 10 is adjustable, for example by adjusting a distance between the objective 40 and the light-transmissive test tube wall 14. Additionally or alternatively, the focus depth may be adjusted by relatively moving two or more lenses or lensing elements within the objective 40.

The beam homogenizer 30 is designed to output a substantially uniform homogenized beam for a Gaussian input beam of the correct diameter. However, the objective 40 typically introduces some spatial non-uniformity. Accordingly, one or more of the stationary optical components, such as the spreading lens 24, collimating lens 26, focusing lens 34, and/or focusing lenses 36 are optionally configured to introduce spatial non-uniformity into the spatial distribution such that the beam when focused by the objective 40 provides substantially uniform static illumination of the microscope field of view. In some contemplated embodiments, this corrective spatial non-uniformity is introduced by one or more dedicated optical components (not shown) that are included in the optical train 22 for that purpose.

The substantially uniform static illumination of the microscope field of view can be used for fluorescence of any fluorescent dye-tagged epithelial cells disposed within the microscope field of view. Additionally, the fluorescent dye typically imparts a lower-intensity background fluorescence to the buffy coat. The fluorescence is captured by the objective 40, and the captured fluorescence 50 (indicated in FIG. 2 by dotted lines) passes through the dichroic mirror 44, and through an optional filter 52 for removing any stray source light, to be imaged by a camera system 56. The camera system 56 may, for example, include a charge coupled device (CCD) camera for acquiring electronic images that can be stored in a computer, memory card, or other non-volatile memory for subsequent image processing.

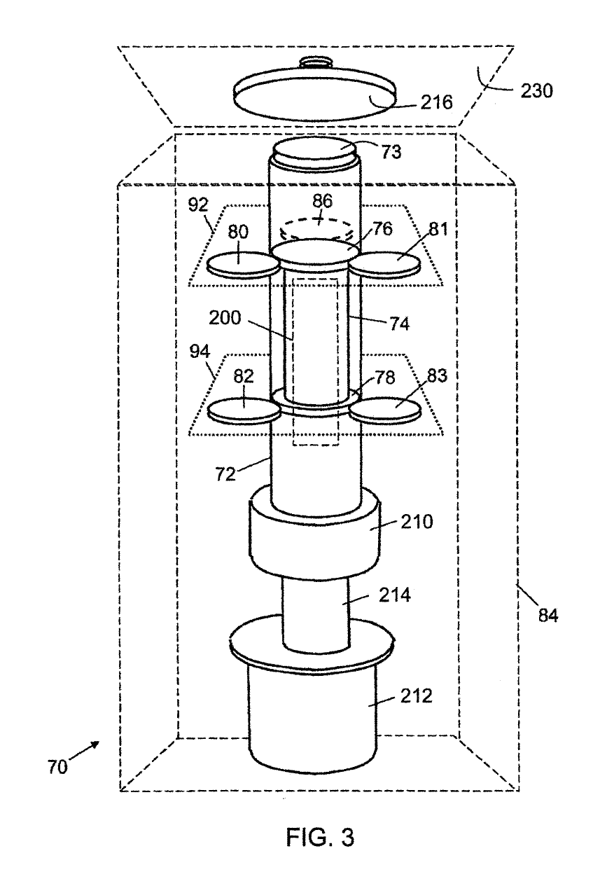

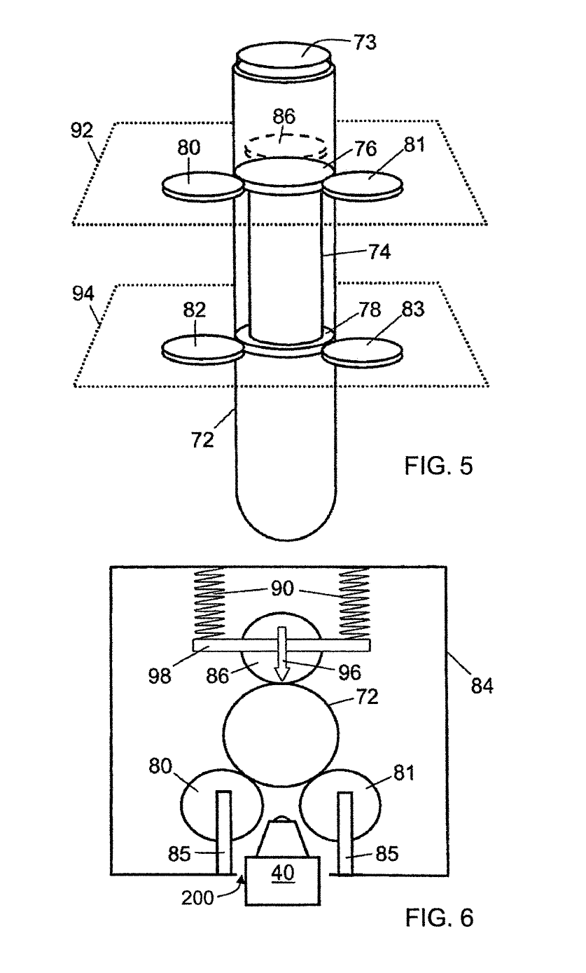

FIGS. 3-6 illustrate a test tube holder to be used with the light system of FIG. 2. A test tube holder 70 has mounted therein a test tube 72 that is sealed by a test tube stopper 73. The sealed test tube 72 contains a float 74 and blood that has been suitably processed and centrifuged to separate out components including red blood cells, plasma, and a buffy coat, as previously described. After centrifuging the float 74 is disposed along the test tube axis 75 (drawn and labeled in FIG. 6). The buffy coat layer may be generally disposed in the annular gap 12 between the test tube wall 14 and the float wall 16. Annular sealing ridges 76, 78 at ends of the float 74 engage an inside surface of the test tube 72 when the test tube is at rest so as to seal the annular gap 12. During centrifuging, however, the test tube 72 expands to provide fluid communication across the ridges 76, 78 so as to enable the buffy coat to substantially collect in the annular gap 12.

At least one first alignment bearing, namely two radially spaced apart first alignment bearings 80, 81 in the example test tube holder 70, are disposed on a first side of the annular sampling region 12. At least one second alignment bearing, namely two second radially spaced apart alignment bearings 82, 83 in the example test tube holder 70, are disposed on a second side of the annular sampling region 12 opposite the first side of the annular sampling region 12 along the test tube axis 75. The alignment bearings 80, 81, 82, 83 are fixed roller bearings fixed to a housing 84 by fastening members 85 (shown only in FIG. 6).

At least one biasing bearing, namely two biasing bearings 86, 87 in the example test tube holder 70, are radially spaced apart from the alignment bearings 80, 81, 82, 83 and are spring biased by springs 90 to press the test tube 72 against the alignment bearings 80, 81, 82, 83 so as to align a side of the annular sampling region 12 proximate to the objective 40 respective to the alignment bearings 80, 81, 82, 83. In the example test tube holder 70, the two first alignment bearings 80, 81 and the first biasing bearing 86 are radially spaced apart by 120° intervals and lie in a first common plane 92 on the first side of the annular sampling region 12. Similarly, the two second alignment bearings 82, 83 and the second biasing bearing 87 are radially spaced apart by 120° intervals and lie in a second common plane 94 on the second side of the annular sampling region 12. The springs 90 are anchored to the housing 84 and connect with the biasing bearings 86, 87 by members 98.

More generally, the bearings 80, 81, 86 and the bearings 82, 83, 87 may have radial spacings other than 120°. For example the biasing bearing 86 may be spaced an equal radial angle away from each of the alignment bearings 80, 81. As a specific example, the biasing bearing 86 may be spaced 135° away from each of the alignment bearings 80, 81, and the two alignment bearings 80, 81 are in this specific example spaced apart by 90°.

Optionally, the first common plane 92 also contains the float ridge 76 so that the bearings 80, 81, 86 press against the test tube 72 at the ridge 76, and similarly the second common plane 94 optionally also contains the float ridge 78 so that the bearings 82, 83, 87 press against the test tube 72 at the ridge 78. This approach reduces a likelihood of distorting the annular sample region 12. The biasing bearings 86, 87 provide a biasing force 96 that biases the test tube 72 against the alignment bearings 80, 81, 82, 83.

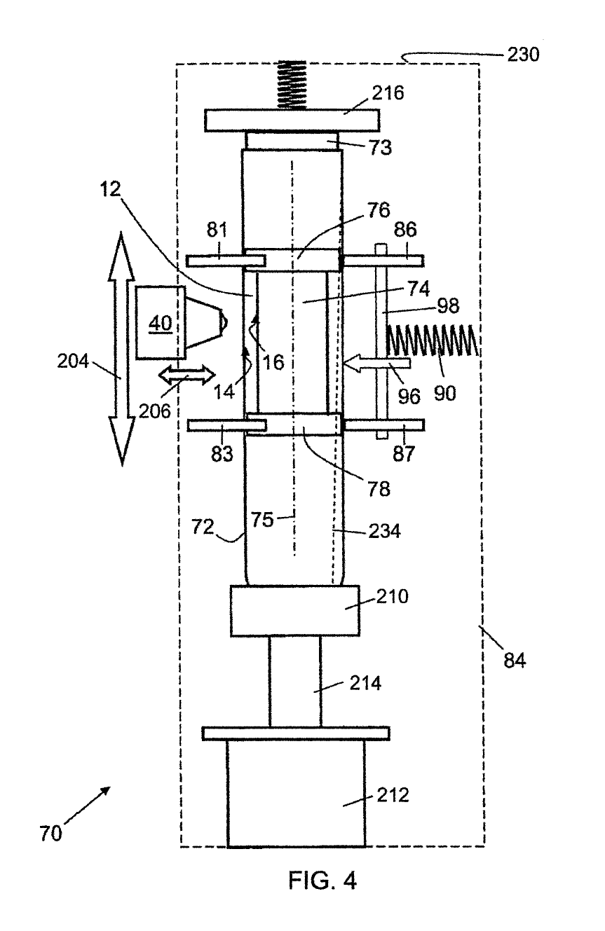

The housing includes a viewing window 200 that is elongated along the tube axis 75. The objective 40 views the side of the annular sample region 12 proximate to the objective 40 through the viewing window 100. In some embodiments, the objective 40 is linearly translatable along the test tube axis 75 as indicated by translation range double-arrow indicator 204. This can be accomplished, for example, by mounting the objective 40 and the optical train 22′ on a common board that is translatable respective to the test tube holder 70. In another approach, the microscope system 10 is stationary, and the tube holder 70 including the housing 84 is translated as a unit to relatively translate the objective 40 across the window 100. In yet other embodiments, the objective 40 translates while the optical train 22 remains stationary, and suitable beam-steering components (not shown) are provided to input the beam to the objective 40. The objective 40 is also focusable, for example by moving the objective 40 toward or away from the test tube 72 over a focusing range 206 (translation range 204 and focusing range 206 indicated only in FIG. 4).

Scanning of the annular sampling region 12 calls for both translation along the test tube axis, and rotation of the test tube 72 about the test tube axis 75. To achieve rotation, a rotational coupling 210 is configured to drive rotation of the test tube 72 about the tube axis 75 responsive to a torque selectively applied by a motor 212 connected with the rotational coupling 210 by a shaft 214. The rotational coupling 210 of the example test tube holder 70 connects with the test tube 72 at an end or base thereof. At an opposite end of the test tube 72, a spring-loaded cap 216 presses against the stopper 73 of the test tube 72 to prevent the rotation from causing concomitant translational slippage of the test tube 72 along the test tube axis 75.

In order to install the test tube 72 in the test tube holder 70, the housing 84 is provided with a hinged lid or door 230 (shown open in FIG. 3 and closed in FIG. 4). When the hinged lid or door 230 is opened, the spring-loaded cap 216 is lifted off of the stopper 73 of the test tube 72. Optionally, the support members 98 that support the biasing bearings 86, 87 include a manual handle or lever (not shown) for manually drawing the biasing bearings 86, 87 away from the test tube 72 against the biasing force of the springs 90 so as to facilitate loading or unloading the test tube 72 from the holder 70.

The test tube holder 70 advantageously can align the illustrated test tube 72 which has straight sides. The test tube holder 70 can also accommodate and align a slightly tapered test tube. The held position of a tapered test tube is indicated in FIG. 4 by a dashed line 234 which indicates the tapered edge of a tapered test tube. The illustrated tapering 234 causes the end of the test tube closest to the rotational coupling 210 to be smaller diameter than the end of the test tube closest to the spring-loaded cap 216. The biasing of the biasing bearings 86, 87 presses the test tube against the alignment bearings 81, 82, 83, 84 to maintain alignment of the portion of the annular sample region 12 proximate to the objective 40 in spite of the tapering 234. It will be appreciated that the holder 70 can similarly accommodate and align a test tube having an opposite taper in which the end closes to the rotational coupling 210 is larger in diameter than the end closest to the spring-loaded cap 216.

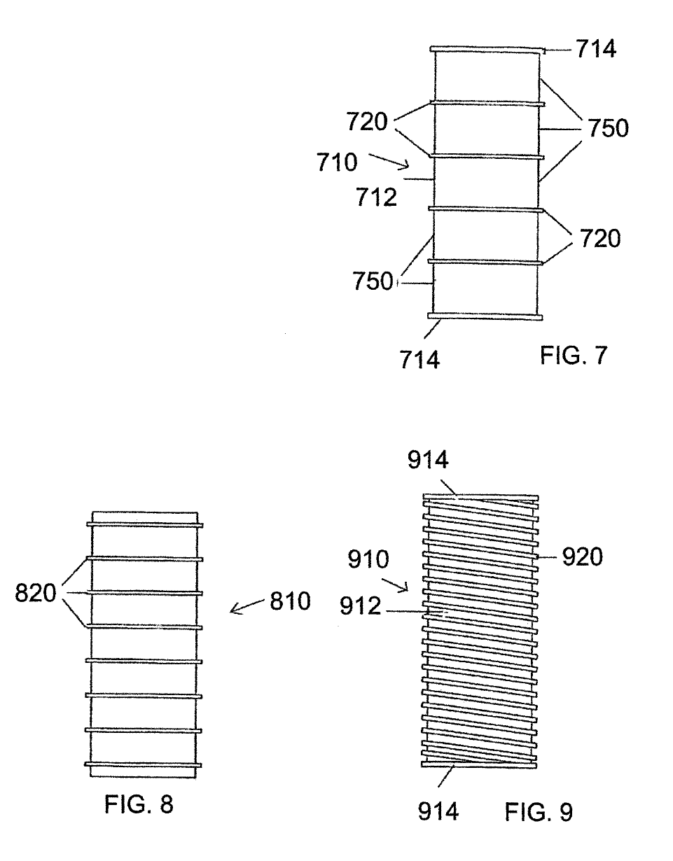

FIGS. 7-22 show various other embodiments of a separator float which can be used in practicing the methods of the present disclosure. These embodiments are also seen in U.S. Pat. No. 7,074,577, the entirety of which is fully incorporated by reference herein.

FIG. 7 illustrates a float 710 according to a further embodiment. The float 710 has a plurality of ribs 720 axially spaced along a central body portion 712, and plural annular channels 750 are defined therebetween. Optional sealing ridges 714 are disposed at opposite ends of the float. Again, the illustrated embodiment depicts continuous ribs, however, it will be recognized that the support ribs may likewise be broken or segmented to provide an enhanced flow path between adjacent annular channels 750.

FIG. 8 illustrates a further float embodiment 810, similar to the embodiment of FIG. 7, the above descriptions of which are equally applicable thereto. However, the float 810 differs in that it lacks sealing ridges at the opposite ends thereof, which may optionally be provided, and the spacing between the ribs 820 is different as well.

FIG. 9 illustrates a further float embodiment 910, wherein a helical support member or ridge 920 is provided. That is, instead of discrete annular bands, multiple turns of the helical ridge 920 provides a series of spaced apart ridges on the main body portion 912, which defines a corresponding helical channel. The helical ridge 920 is illustrated as continuous, however, the helical band may instead be segmented or broken into two or more segments, e.g., to provide path for fluid flow between adjacent turns of the helical buffy coat retention channel. Optional sealing ridges 914 appear at each axial end of the float 910.

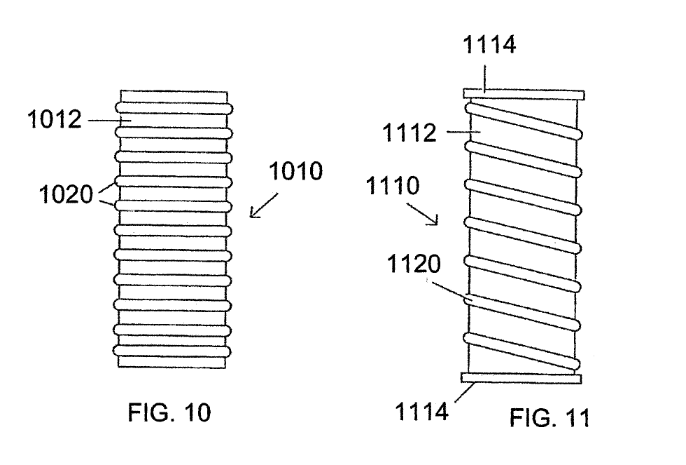

FIG. 10 illustrates another ribbed embodiment 1010. Radial support members 1020 extend radially from the main body portion 1012. The support members 1020 each have a generally curved or rounded cross-sectional profile. Again, the support members 1020 are shown as continuous but may, in alternative embodiments, be discontinuous or segmented. End sealing ridges are not present in FIG. 10, but may optionally be provided.

FIG. 11 illustrates another embodiment of a separator float 1110. Here, the support member 1120 is helical, and extends from main body portion 1112. End sealing ridges 1114 are present, though again they are optional.

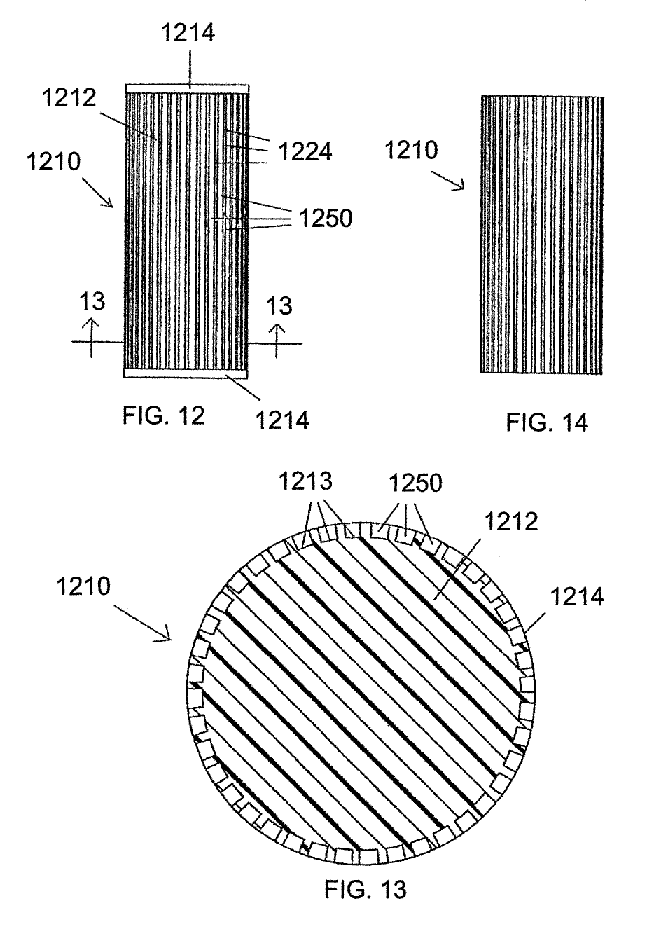

Referring now to FIG. 12 and FIG. 13, there is shown a splined separator float 1210. The float 1210 includes a plurality of axially-oriented splines or ridges 1224 radially spaced about a central body portion 1212. Optional end sealing ridges 1214 are disposed at opposite ends of the float. The splines 1224 and the optional end sealing ridges 1214 protrude from the main body 1212 to engage and provide support for the deformable tube. Where provided, the end sealing ridges 1214 provide a sealing function as described above. The axial protrusions 1224 define fluid retention channels 1250, between the tube inner wall and the main body portion 1212. The surfaces 1213 of the main body portion disposed between the protrusions 1224 may be curved, e.g., when the main body portion is cylindrical, however, flat surfaces 1213 are also contemplated. Although the illustrated embodiment depicts splines 1224 that are continuous along the entire axial length of the float, segmented or discontinuous splines are also contemplated.

FIG. 14 illustrates an embodiment of the float 1210 wherein the end sealing ridges are not provided.

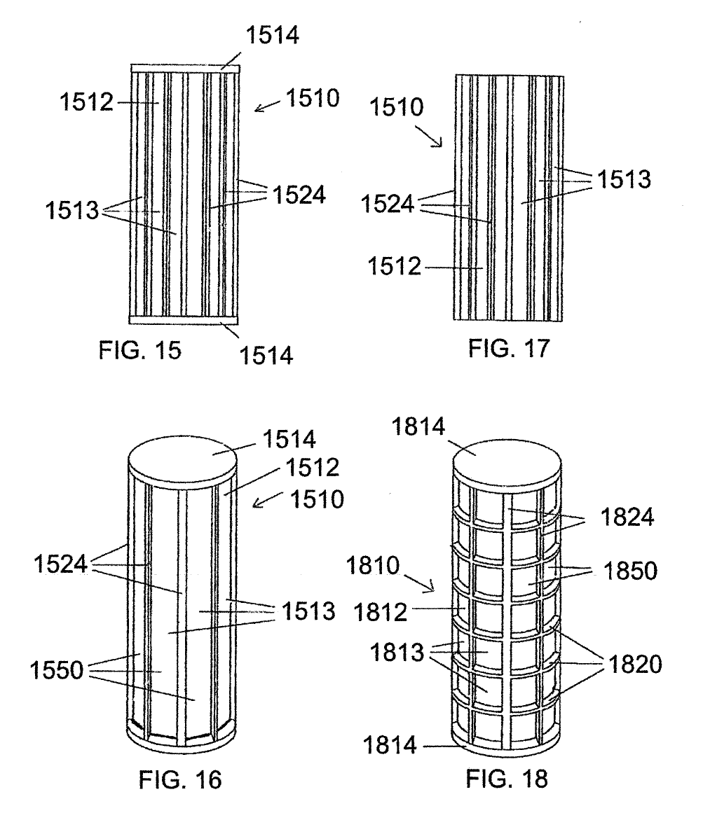

FIG. 15 is a side view of another embodiment of the float 1510, while FIG. 16 is a perspective view of the float. Here, axially aligned ribs or splines 1524 protrude from the main body portion 1512. The float 1510 includes optional end sealing ridges 1514 which are radially aligned and are disposed at opposite ends of the float 1510. Fluid retention channels 1550 formed between adjacent splines 1524 are defined by adjacent splines 1524 and surfaces 1513 on the main body portion 1512. The surfaces 1513 are depicted as generally flat, although curved surfaces are also contemplated. The axial splines 1524 are continuous along the length of the tube; however, segmented or discontinuous splines are also contemplated.

FIG. 17 illustrates an embodiment of the float 1510 wherein the end sealing ridges are not provided.

Referring now to FIG. 18, there is shown yet another embodiment 1810. The support members. The support means 1820 can be described as an intersecting network of annular rings or ribs 1826 and axial splines 1824. Optional end sealing ridges 1814 are disposed at opposite ends of the float. The support members 1820 and the optional sealing ridges 1814 engage and provide support for the deformable tube. Where provided, the end sealing ridges 1814 provide a sealing function as described above. The raised support members 1820 define a plurality of fluid retention windows 1850 formed between the tube inner wall and the main body portion 1812. Surfaces 1813 of the main body portion 1812 corresponding to the windows 1850 may be curved, e.g., when the main body portion is cylindrical, however, flat surfaces are also contemplated. Although the illustrated embodiment depicts the support members 1820 as a network of annular ribs and axial splines which is continuous, breaks may also be includes in the annular and/or axial portions of the network 1820, e.g., to provide a fluid path between two or more of the windows 1850.

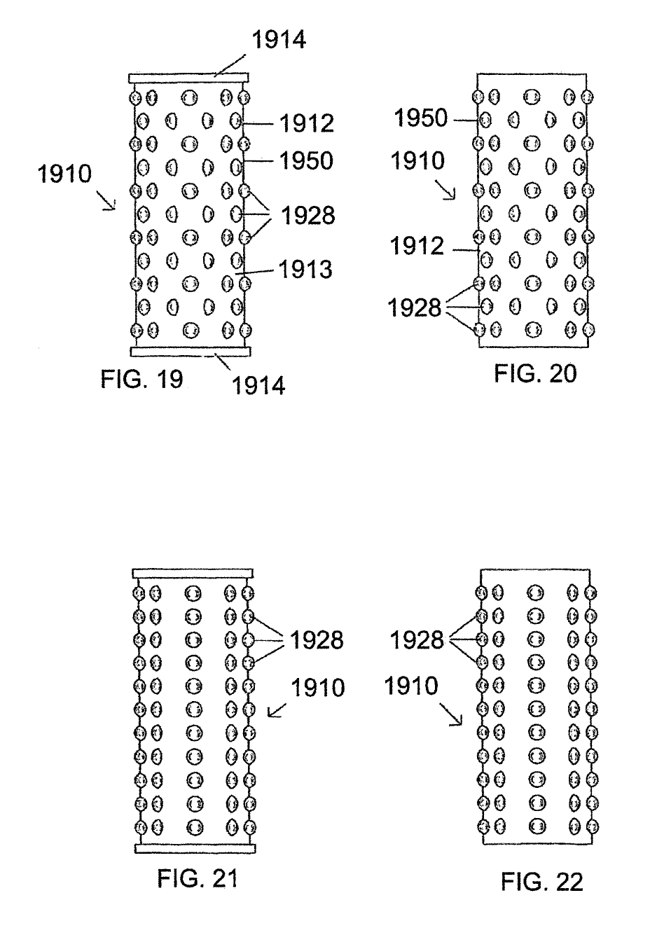

FIGS. 19-22 illustrate several floats having a plurality of protrusions thereon for providing support for the deformable walls of the sample tube.

Referring to FIG. 19, the float 1910 includes multiple rounded protrusions 1928 spaced over the surface 1913 of the central body portion 1912 in a staggered pattern. Optional end sealing ridges 1914 are disposed at opposite ends of the float 1910. The protrusions 1928 and the optional end sealing ridges 1914 radially protrude from the main body 1912 and traverse an annular gap 1950 to engage and provide support for the deformable tube wall. When provided, the end sealing ridges 1914 provide a sealing function as described above. The surface 1913 of the main body portion disposed between the protrusions may be curved, e.g., when the main body portion is cylindrical, or, alternatively, may have flat portions or facets.

FIG. 20 illustrates an embodiment of the float 1910 wherein the end sealing ridges are not provided.

In FIG. 21, the protrusions 1928 are spaced over the surface in an aligned pattern. End sealing ridges 1914 are provided.

In FIG. 22, the protrusions 1928 are spaced over the surface in an aligned pattern, and end sealing ridges are absent.

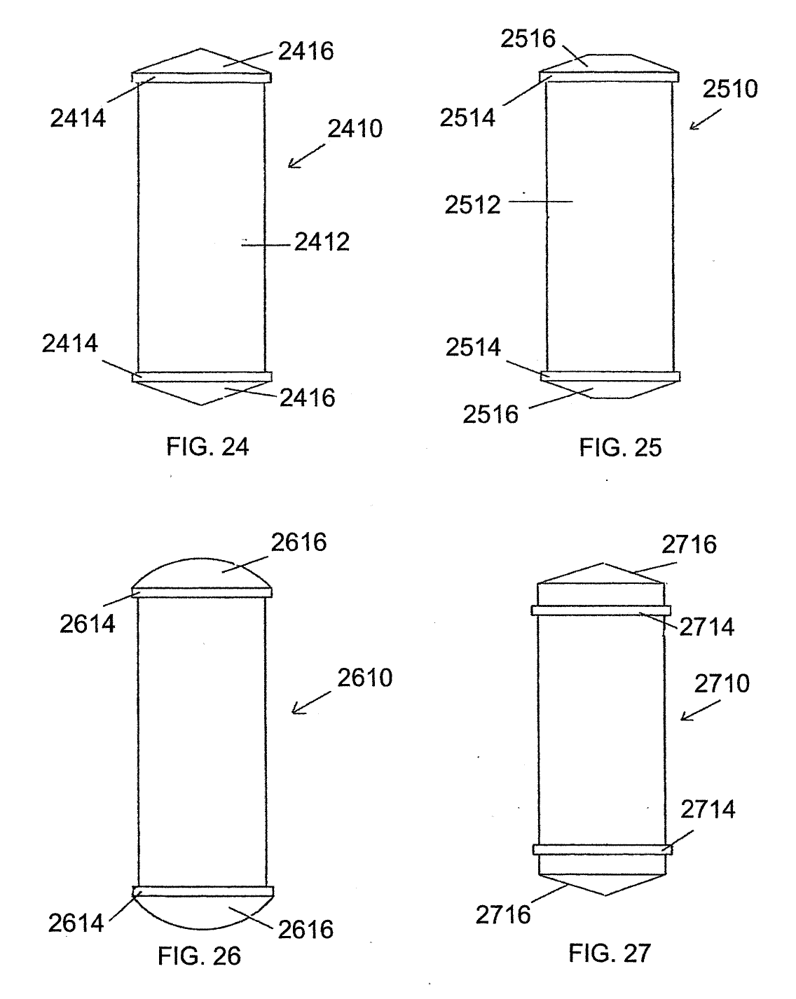

Additional embodiments of separator floats are shown in FIGS. 24-29. These embodiments are also seen in U.S. Pat. No. 7,220,593, the entirety of which is fully incorporated by reference herein.

FIG. 24 illustrates a float 2410 that includes a main body portion 2412 and sealing rings 2414. The ends of the main body portion may be considered as including a tapered or cone-shaped endcap member 2416 disposed at each end. The tapered endcaps 2416 are provided to facilitate and direct the flow of cells past the float 2410 and sealing ridges 2414 during centrifugation.

FIG. 25 illustrates a float 2510 that includes a main body portion 2512 and sealing ridges 2514 similar to FIG. 24. Here, the endcap members 2516, disposed at each end, have a frustoconical shape.

FIG. 26 illustrates a float 2610 having generally convex or dome-shaped endcap members 2616, which cap the sealing ridges 2614. The endcaps 2616 may be hemispherical, hemiellipsoidal, or otherwise similarly sloped, are provided. Again, the sloping ends 2616 are provided to facilitate density-motivated cell and float movement during centrifugation.

The geometrical configurations of the endcap units 2416, 2516, and 2616 illustrated in FIGS. 24-26, respectively, are intended to be exemplary and illustrative only, and many other geometrical shapes (including concave or convex configurations) providing a curved, sloping, and/or tapered surface around which the blood sample may flow during centrifugation. Additional exemplary shapes contemplated include, but are not limited to tectiform and truncated tectiform; three, four, or more sided pyramidal and truncated pyramidal, ogival or truncated ogival; geodesic shapes, and the like.

FIG. 27 illustrates a float 2710 wherein the sealing ridges are 2714 are axially displaced from the ends. Optional endcap members 2716 appear as conical in the illustrated embodiment. However, it will be recognized that the endcaps 2716, if present, any other geometrical configuration which provides a sloped or tapered surface may be used, as described above.

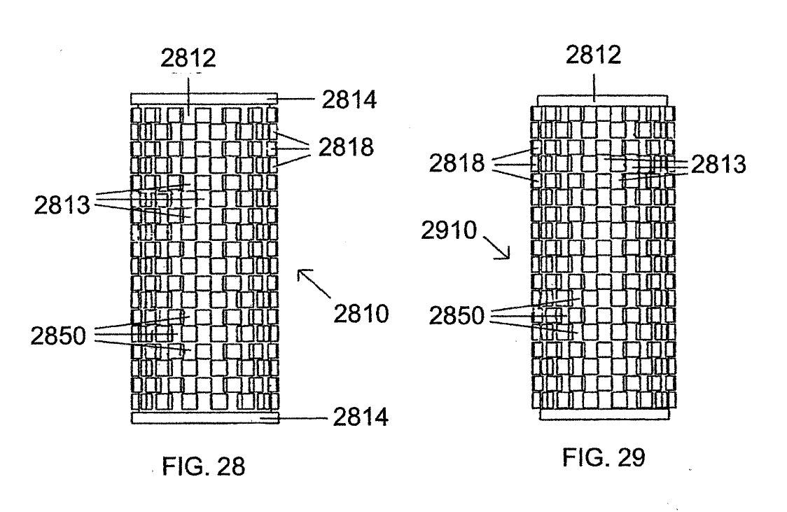

FIG. 28 and FIG. 29 illustrate float embodiments 2810 and 2910, respectively, which include multiple raised facets 2828 spaced over the surface of a main or central body portion 2812. Optional end sealing ridges 2814 are present in FIG. 28, but not FIG. 29. The facets 2828 and the optional end sealing ridges 2814 radially protrude from the main body 2812 and traverse an annular gap to engage and provide support for the test tube sidewall and define a plurality of fluid retention windows 2850. Where provided, the end sealing ridges 2814 provide a sealing function as described above. The surfaces 2813 of the main body portion, disposed between the protrusions 2828 and forming a surface defining the fluid-retention windows 2850, may be curved surfaces, e.g., when the main body portion is cylindrical. Alternatively, the surfaces 2813 may be flat. In alternative embodiments, the size, spacing density, and alignment patterns of the facets 2818 can be modified extensively.

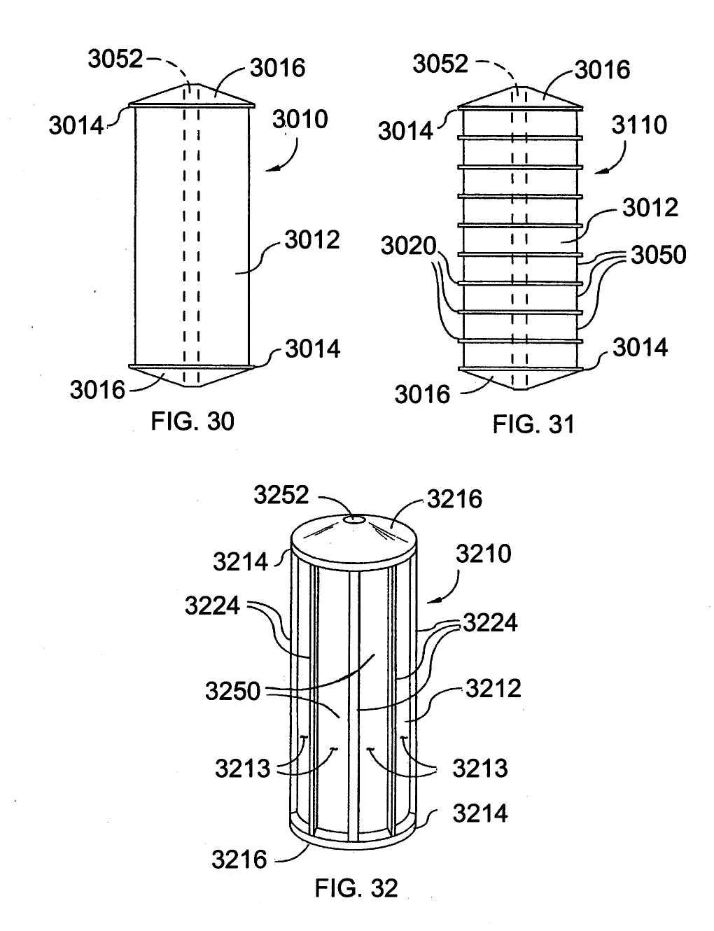

FIG. 30 and FIG. 31 illustrate float embodiments 3010 and 3110, respectively. The main body portion 3012 has a diameter that is smaller than the inner diameter of the test tube. Optionally tapered ends 3016 are provided to facilitate and direct the flow of cells past the float 3010 and sealing ridges 3014 during centrifugation. A central bore 3052, shown in broken lines, provides a pressure relief outlet to alleviate any pressure build up in the lower fluid layers due to the contraction of the tube walls. FIG. 31 includes radially extending ribs 3020 spaced along the axial direction of the main body portion between the two ends of the float. Multiple annular channels 3050 are defined between the main body portion 3012 and the inner tube wall. Although the illustrated embodiment depicts continuous ribs, it will be recognized that the support ribs may likewise be broken or segmented to provide an enhanced flow path between adjacent annular channels 3050.

FIG. 32 shows a splined separator float 3210, including a plurality of axially oriented splines or ridges 3224 which are radially spaced about a central body portion 3212. End sealing ridges 3214 and optionally tapered ends 3216 are provided to facilitate and direct the flow of cells past the float 3210 and sealing ridges 3214 during centrifugation. The splines 3224 and the end sealing ridges 3214 protrude from the main body 3212 to engage and provide support for the deformable tube once centrifugation is completed. The axial protrusions 3224 define fluid retention channels 3250, between the tube inner wall and the main body portion 3212. The surfaces 3213 of the main body portion disposed between the protrusions 3224 may be curved, e.g., when the main body portion 3212 is cylindrical, however, flat surfaces 3213 are also contemplated. Although the illustrated embodiment depicts splines 3224 that are continuous along the entire axial length of the float 3210, segmented or discontinuous splines are also contemplated. A pressure relief bore 3252 extends axially and centrally through the float 3210. In other embodiments, one or more of such pressure relief bores, of similar or different shape, can be included in the main body of the float.

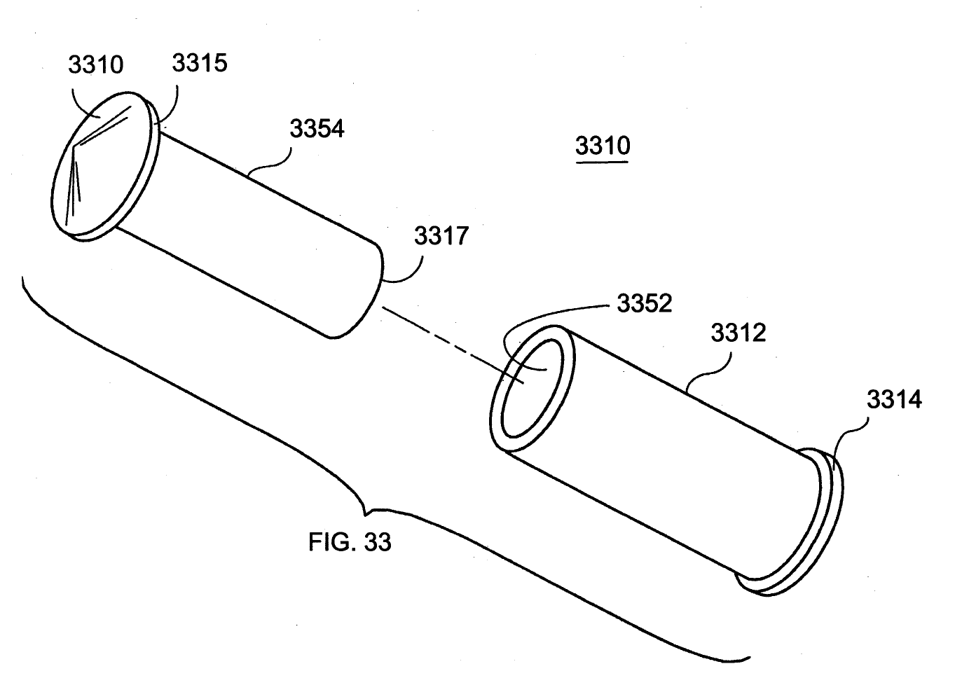

FIG. 33 illustrates a two-piece float 3310 in accordance with a preferred embodiment of the present disclosure, shown in exploded view. A first, main body portion or sleeve 3312 includes a central bore 3352, which is sized to slidably receive a second, piston-like center portion 3354. The outer body member 3312 includes a flange or sealing ring 3314, which is at its lower or bottom end. A sealing ridge or flange 3315 is disposed at the upper end of the piston section 3354 during operation. Optionally tapered ends 3317 are preferably provided at the upper and lower (during operation) ends of the piston portion 3354 to facilitate and direct the flow of cells past the sealing ridges 3314 and 3315 during centrifugation.

In operation, the piston portion 3354 is fully received within the central bore 3352 of the main body member 3312. As stated above, the float 3310 is oriented in the tube so that the sealing ridge 3315 is at the top and the sealing ridge 3314 is toward the bottom of the tube. The two portions may be formed of the same material or different materials, so long as the overall specific gravity of the float 3310 is in a suitable range for buffy coat capture. In an especially preferred embodiment, the central piston portion 3354 is formed of a slightly higher specific gravity material than the outer portion 3312, which insures that the two portions stay together during centrifugation. Alternatively, the two float members are formed of the same material and/or a frictional fit sufficient to keep the float members together during centrifugation is provided.

As the tube containing the blood sample and float 3310 is centrifuged, the two pieces 3312 and 3354 stay together and act in the same manner as a one-piece float to axially expand the buffy coat layers. When separation and layering of the blood components is complete and centrifugation is slowed, pressure may build in the red blood cell fraction trapped below the float, e.g., where contraction of the tube continues after initial capture of the float by the tube wall. Any such pressure in the trapped red blood cell region forces the center piece 3354 upward, thus relieving the pressure, and thereby preventing the red blood cells from breeching the seal between the sealing rings 3314 and the tube wall.

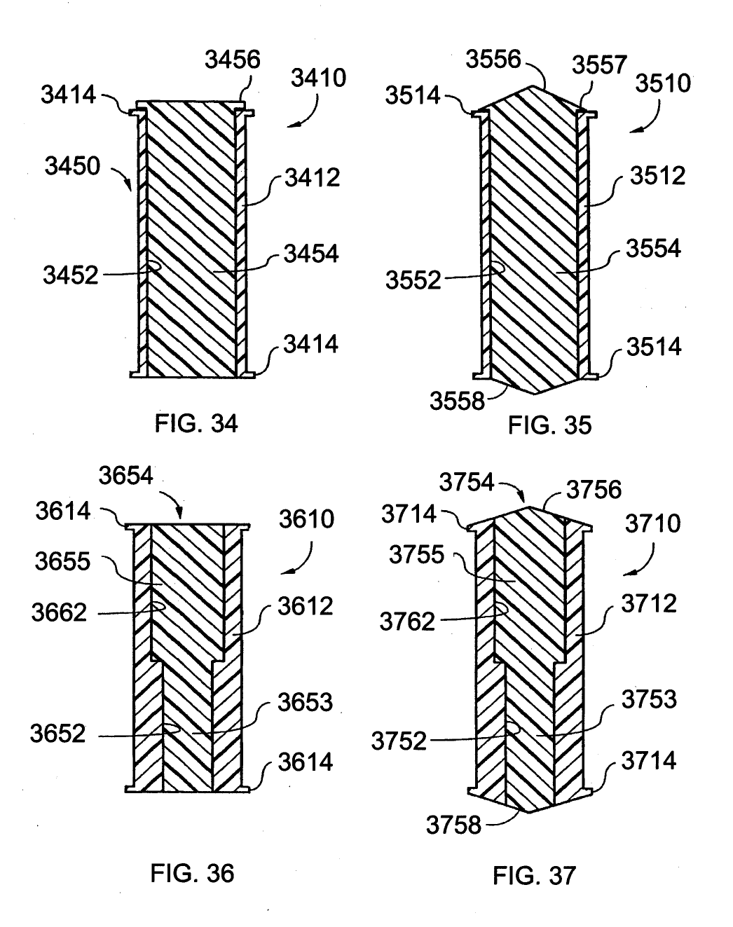

FIGS. 34-40 illustrate further two-piece float embodiments of the present disclosure wherein the sealing rings are disposed at each end of the outer sleeve and pressure relief is provided by an upwardly movable piston member.

FIG. 34 illustrates a two-piece float 3410 including a first, main body portion or sleeve 3412 having a central bore 3452 slidably receiving a second, piston-like center portion 3454. The outer body member 3412 includes a sealing ring or ridge 3414 at each end sized to engage the test tube sidewall, with an annular recess 3450 defined therebetween. The piston 3454 includes a flanged end 3456 that is greater in diameter than the central bore 3452 and less than the diameter of the sealing ridges 3414.

In operation, the piston member 3454 is fully received within the central bore 3452, with the flange 3456 abutting the upper end of the sleeve 3412. In use, the float 3410 is oriented in the tube so that the flange 3456 is located toward the top of the test tube 130, i.e., toward the stopper 140 (FIG. 1). Again, the two portions may be formed of the same material or different materials, so long as the overall specific gravity of the float 3410 is in a suitable range for buffy coat capture. In an especially preferred embodiment, the central portion 3454 is formed of a slightly higher specific gravity material than the outer portion 3412, which insures that the two portions stay together during centrifugation. Alternatively or additionally, a frictional fit is provided between the two float sections. Upon completion of centrifugation, any pressure build up in the trapped red blood cell region is alleviated by forcing the center piece 3454 upwardly.

FIG. 35 illustrates a two-piece float 3510 similar to that shown and described by way of reference to FIG. 34, but further including tapered ends for facilitating blood flow around float 3510 during centrifugation. A first, main body portion or sleeve 3512 has a central bore 3552 slidably receiving a second, piston-like center portion 3554. The outer body member 3512 includes sealing rings or ridges 3514 at opposite ends, as described above. The piston 3554 includes a tapered end 3556 including a flange 3557 sized to abut the sleeve 3512 upon insertion and restrict any further downward passage of the piston 3554. A lower end 3558 of the piston member 3554 is also tapered to facilitate flow. Centrifugal motivation and/or a frictional fit may be used to insure the two sections remain together during centrifugation.

FIG. 36 illustrates a two-piece float 3610 including a first, main body portion or sleeve 3612 having a central bore 3652 and a counterbore 3662, slidably receiving a second, piston-like center portion 3654. The outer body member 3612 includes a sealing ring or ridge 3614 as described above. The piston 3654 includes a first, smaller diameter portion sized to be received within the central bore 3652 and a second, larger diameter portion sized to be received within the counterbore 3662. The axial extent of the small diameter segment 3653 and large diameter segment 3655 may vary widely and are complimentary to that of the bore 3652 and counterbore 3662, respectively. Although the float 3610 is shown with generally flat ends, it will be recognized that the ends of the piston member 3654 and/or sleeve member 3612 may be tapered to facilitate fluid flow around the float during centrifugation.

FIG. 37 illustrates an embodiment similar to that shown in FIG. 36, having tapered ends. A two-piece float 3710 includes a first, main body portion or sleeve 3712 having a central bore 3752 and a counterbore 3762, slidably receiving a second, piston-like center portion 3754. The outer body member 3712 includes a sealing ring or ridge 3714. The piston 3754 includes a first, smaller diameter portion sized to be received within the central bore 3752 and a second, larger diameter portion sized to be received within the counterbore 3762. The tapered ends 3756 and 3758 cooperate with complimentary end ridges to form generally conical ends.

Referring to FIG. 36 and FIG. 37, during centrifugation, the float is oriented in the tube so that the counterbore and larger diameter portion are located toward the top of the test tube. As described above, the two portions may be formed of the same material or different materials and, in the preferred embodiment, the central portion (3654; 3754) is formed of a slightly higher specific gravity material than the outer sleeve (3612; 3712) insuring that the two sections stay together during centrifugation. Upon completion of centrifugation, any pressure built up in the trapped red blood cell region forces the center section (3654; 3754) upwardly.

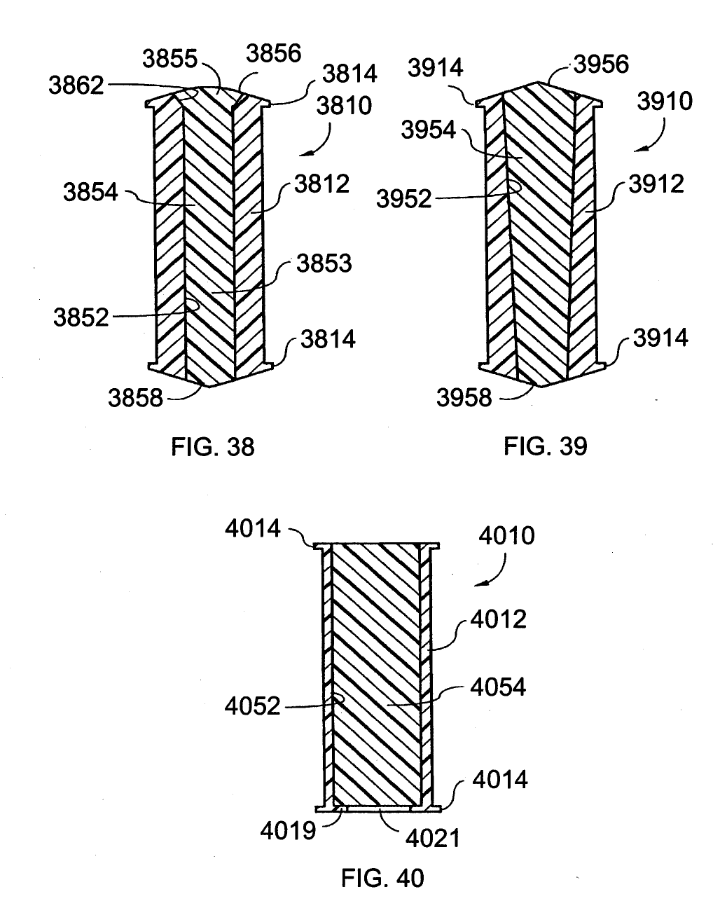

FIG. 38 illustrates yet another two-piece float embodiment 3810 including a first, main body portion or sleeve 3812 having a profiled bore comprising a central bore 3852 and an enlargement or countersink 3862 opening toward the upper end of the tube. A second, piston-like movable member 3854 includes a shaft 3853 and an enlarged head 3855, which are complimentary to and slidably received in the central bore 3852 and the countersink 3862, respectively. The outer sleeve 3812 includes sealing rings or ridges 3814 as described above. The float 3810 is shown with tapered ends 3856 and 3858, however, it will be recognized that the ends of the float 3810 may also be flat. As described above, the two sections 3812 and 3854 may be formed of the same material or different materials and, in the preferred embodiment, the movable member 3854 is formed of a slightly higher specific gravity material than the outer sleeve 3812, insuring that the two sections stay together during centrifugation.

FIG. 39 illustrates a further two-piece separator float embodiment 3910 including a first, main body portion or sleeve 3912 having a tapered internal passage 3952 which widens toward the upper end 3956 of the float. A central, movable member 3954 complimentary to the bore 3952 is slidably received therein. The outer sleeve 3912 includes sealing rings or ridges 3914. The separator float ends 3956 and 3958 are illustrated as tapered, although flat ends are also contemplated. The two sections 3912 and 3954 may be formed of the same material or different materials, again, with the movable member 3954 preferably formed of a slightly higher specific gravity material to keep the float sections together during centrifugation.

FIG. 40 illustrates a further two-piece separator float embodiment 4010 including a first, main body portion or sleeve 4012 having an central passage or bore 4052 which terminates in an annular seat 4019 formed at a lower end of the float 4010 and defining an opening 4021 into the bore 4052. A piston-like movable member 4054 is slidably received within the bore 4052, abutting the annular seat 4019. The outer sleeve 4012 includes sealing rings or ridges 4014. The separator float 4010 is depicted with flat ends, although tapered ends are also contemplated. Optionally, the movable member 4054 may contain a narrow diameter portion (not shown) on the lower end thereof sized to be received in the aperture 4021, e.g., to provide a flush and/or tapered surface to facilitate flow therepast during centrifugation. The two sections 4012 and 4054 may be formed of the same material or different materials; preferably, the movable member 4054 is formed of a slightly higher specific gravity material to keep the float sections together during centrifugation.

Each of the float embodiments of FIGS. 30-40, which have been illustrated with end sealing rings and without additional tube supporting members for ease of demonstration, may be further modified by the further incorporation of any of the tube support features as shown in the earlier figures, such as annular bands, segmented bands, helical bands, axial splines, rounded protrusions, spikes, facets, and combinations thereof. Likewise, the separator float embodiments are depicted herein having either flat or the preferred conical ends; however, many other geometrical shapes providing a curved, sloping, and/or tapered surface to facilitate density-motivated cell and float movement during centrifugation are contemplated. Exemplary modified end shapes include, for example, frustoconical, convex or dome-shaped, and other tapered shapes.

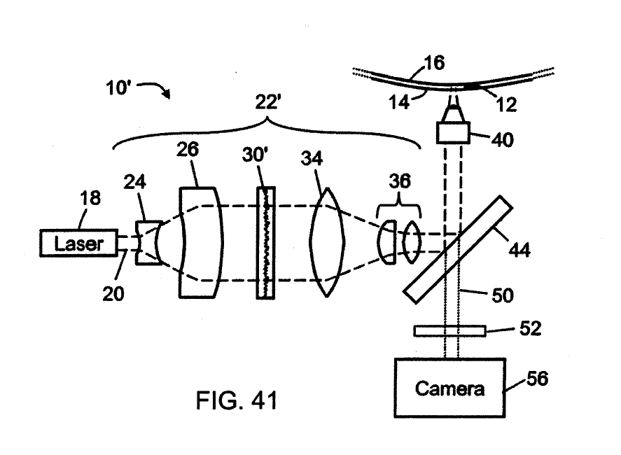

Referring back to FIG. 2, some variations that lead to other suitable microscope systems are described in FIGS. 41-43. FIG. 41 shows a microscope system 10′ that is similar to the microscope system 10 of FIG. 2, except that the optical train 22′ differs in that the stationary beam homogenizer 30 of FIG. 2 is replaced by a stationary diffuser 30′. The diffuser 30′ may, for example, be a holographic diffuser available, for example, from Physical Optics Corporation (Torrance, Calif.). Such holographic diffusers employ a hologram providing randomizing non-periodic optical structures that diffuse the light to impart improved spatial uniformity. However, the diffusion of the light also imparts some concomitant beam divergence. Typically, stronger diffusion of the light tend to impart more spatial uniformity, but also tends to produce greater beam divergence. Holographic diffusers are suitably classified according to the full-width-at-half-maximum (FWHM) of the divergence angle, with larger divergence angles typically providing more diffusion and greater light uniformity, but also leading to increased light loss in the microscope system 10′ due to increased beam divergence.

In some embodiments of the microscope system 10′, the diffuser 30′ is a low-angle diffuser having a FWHM less than or about 10°. Lower angle diffusers are generally preferred to provide less divergence and hence better illumination throughput efficiency; however, if the divergence FWHM is too low, the diffuser will not provide enough light diffusion to impart adequate beam uniformity. Low diffusion reduces the ability of the diffuser 30′ to homogenize the Gaussian distribution, and also reduces the ability of the diffuser 30′ to remove speckle.

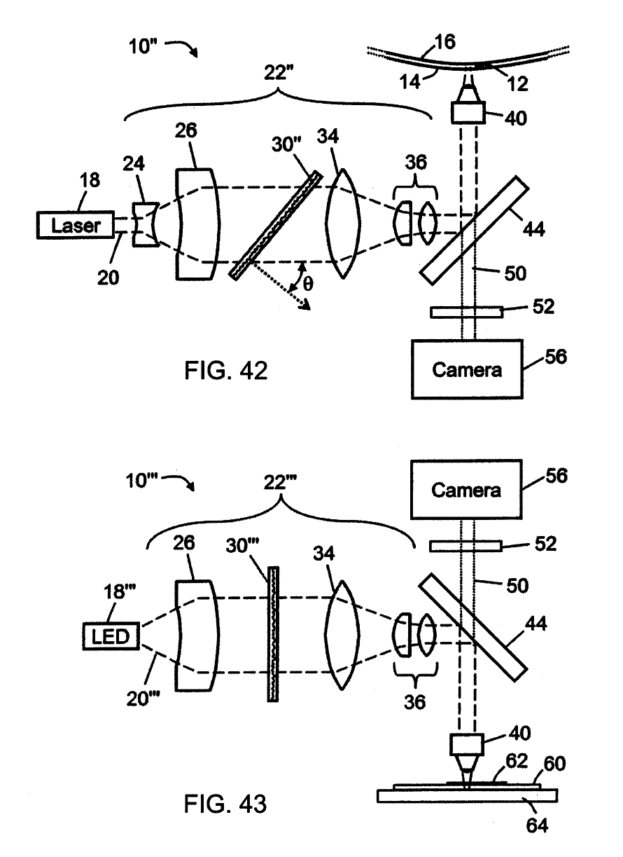

With reference to FIG. 42, another embodiment microscope system 10″ is similar to the microscope system 10′, and includes an optical train 22″ that employs a diffuser 30″ similar to the diffuser 30′ of the microscope system 10′. However, the diffuser 30″ is tilted at an angle θ respective to the optical path of the optical train 22″ so as to substantially reduce a speckle pattern of the source light 20. Without being limited to any particular theory of operation, it is believed that the tilting shifts the speckle pattern to higher spatial frequencies, in effect making the speckle size smaller. The speckle size is spatially shifted by the tilting such that the frequency-shifted speckle is substantially smaller than an imaging pixel size.

In some embodiments, a tilt angle θ of at least about 30° respective to the optical path of the optical train 22″ is employed, which has been found to substantially reduce speckle for diffusers 30″ having a FWHM as low as about 5°. On the other hand, tilt angles θ of greater than about 45° have been found to reduce illumination throughput efficiency due to increased scattering, even for a low-angle diffuser having a FWHM of 5°.

In FIG. 43, a microscope system 10′″ includes a light emitting diode (LED) 1′″ as the light source, rather than the laser 18 used in the previous microscope systems 10, 10′, 10″. Because the LED 18′″ outputs diverging source light 20′″ rather than a collimated laser beam, an optical train 22′″ is modified in that the beam-expanding concave lens 24 is suitably omitted, as shown in FIG. 43. Alternatively, a lens can be included in the position of the lens 24, but selected to provide a suitable divergence angle adjustment for collimation by the collimating lens 26. The optical train 10′″ employs a diffuser 30′″ similar to the diffusers 30′, 30″. The LED 18′″ outputs incoherent light, and so speckle is generally not present. However, the output of the LED 18′″ typically does have a non-Gaussian distribution, for example a Lambertian distribution. In view of these characteristics of the source light 20′″, the diffuser 30′″ is not tilted, and in some cases the diffuser 30′″ can have a smaller divergence angle FWHM than the unfitted diffuser 30′ used to impart spatial uniformity to the laser beam source light 20 in the microscope system 10′ of FIG. 41.

The microscope system 10′″ of FIG. 43 further differs from the microscope systems 10, 10′, 10″ in that the microscope system 10′″ images a sample disposed on a planar slide 60, which is optionally covered by an optional cover glass 62. The slide 60 is disposed on an x-y planar translation stage 64 to enable scanning across the sample. It will be appreciated that the LED 18′″ and optical train 22′″ are also suitable for imaging the buffy coat sample disposed in the annular gap 12 between the light-transmissive test tube wall 14 and float wall 16 shown in FIG. 41 and FIG. 42. Conversely, it will be appreciated that the laser 18 and optical train 22, 22′, 22″ are also suitable for imaging the planar sample on the slide 60 shown in FIG. 43.

The optical trains 22, 22′, 22″, 22′″ have components which are stationary in the sense that the components are not rotated, relatively oscillated, or otherwise relatively moved. It is, however, contemplated to move the optical train and the objective 40 as a whole, and/or to include beam-steering elements, or so forth, to enable relative scanning of the field of view respective to the sample.

Suitable microscope systems for imaging an annular sample contained in or supported by a test tube have been described in FIG. 2 and FIGS. 41-43. The annular gap 12 typically has a thickness that is substantially larger than a depth of view of the microscope objective 40. The test tube wall 12 and float wall 16 are typically not uniform across the entire surface of the test tube or float. While the microscope objective 40 typically has an adjustable depth of focus (adjusted by moving internal optical components and/or by moving the objective 40 toward or away from the test tube wall 12), the range of adjustment is limited. Accordingly, the test tube should be held such that the surface proximate to the objective 40 is at a well-defined distance away from the objective 40 as the test tube is rotated and as the objective 40, or the test tube, is translated along a tube axis.

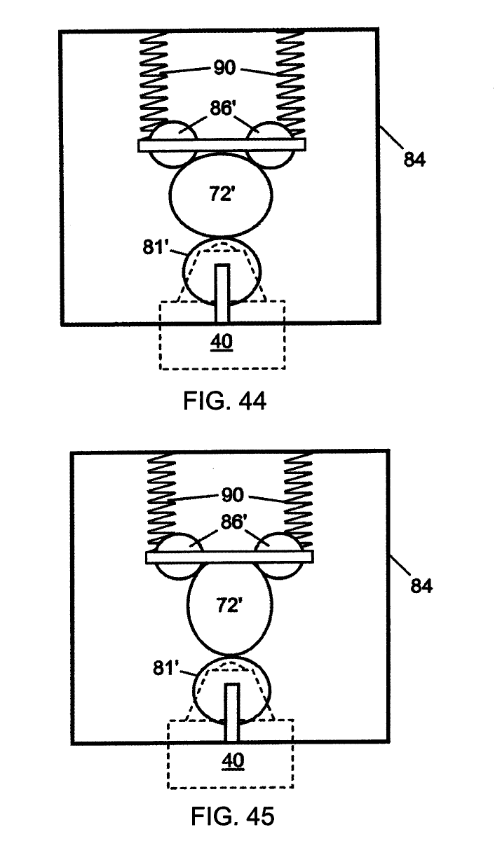

A variation on the test tube holder of FIGS. 3-6 is shown in FIG. 44 and FIG. 45. A modified test tube 72′ having an elliptical cross section is more precisely aligned by employing a set of three bearings per supported float ridge,in which the three bearings include only one alignment bearing 81′ and two or more biasing bearings 86′. The alignment bearing 81′ is at the same radial position as the objective 40 (shown in phantom in FIG. 44 and FIG. 45). As the elliptical test tube 12′ rotates, the imaged side that is biased against the alignment bearings 81′ remains precisely aligned with the radially coincident objective 40 whether the imaged side correspond to the short axis of the elliptical test tube 72′ (FIG. 44), or whether the imaged side correspond to the long axis of the elliptical test tube 72′ (FIG. 45).

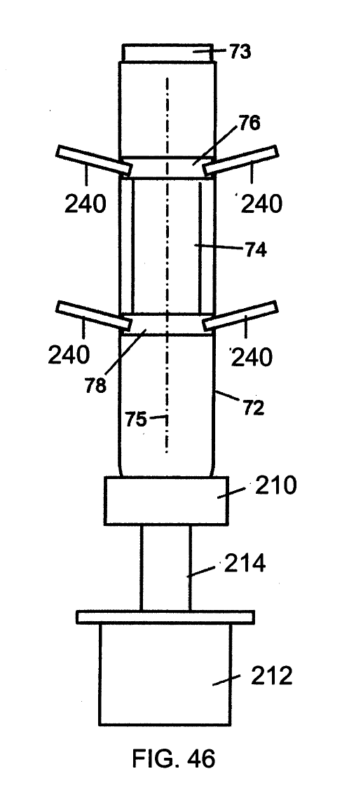

With reference to FIG. 46, in another variation, bearings 240 are tilted respective to the tube axis 75 of the test tube 72 to impart force components parallel with the tube axis 75 to push the test tube 72 into the rotational coupling 210. In this arrangement, the spring loaded cap 216 is optionally omitted, because the tilting of the bearings 240 opposes translational slippage of the test tube 72 during rotation.

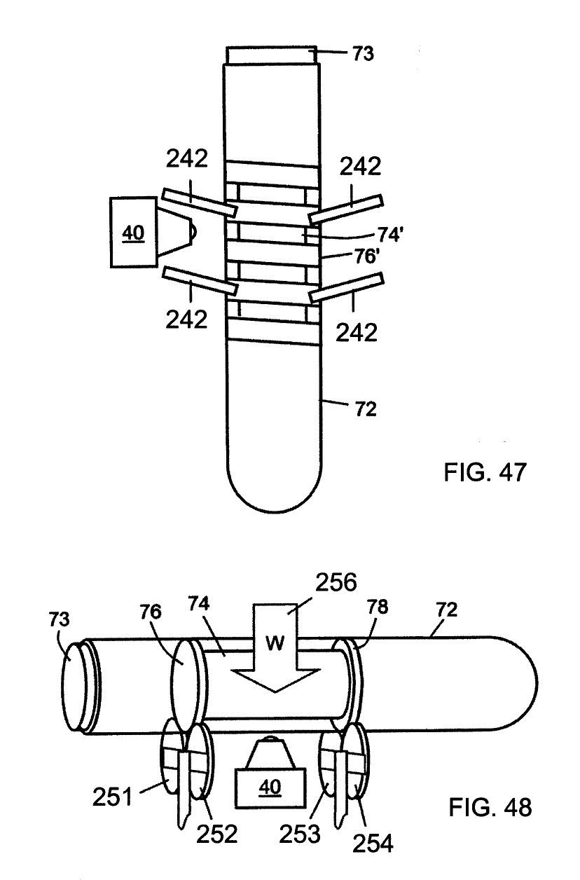

With reference to FIG. 47, in another variation, a modified float 74′ includes spiral ridges 76′, and tilted bearings 242 are spaced along the tube axis 75 in accordance with the spiral pitch to track the spiraling sealing ridges 76′ responsive to rotation of the test tube 72. In this approach, the tilted bearings 242 impart a force that causes the test tube 72 to translate along the tube axis 75, so that the objective 40 can be maintained at a fixed position without translating while scanning annular gap 12′. In this approach, the roller bearings 242 are suitably motorized to generate rotation of the test tube 72. That is, the roller bearings 242 also serve as the rotational coupling.

With reference to FIG. 48, in another variation, the mechanical bias can be provided by a mechanism other than biasing bearings. Here, the test tube 72 is arranged horizontally resting on alignment bearings 251, 252, 253, 254 with the objective 40 mounted beneath the test tube 72. A weight of the test tube 72 including the float 74 (said weight diagrammatically indicated by a downward arrow 256) provides as the mechanical bias pressing the test tube 72 against the alignment bearings 251, 252, 253, 254. In other contemplated embodiments, a vacuum chuck, positive air pressure, magnetic attraction, or other mechanical bias is employed to press the test tube against the alignment bearings. The alignment bearings 251, 252, 253, 254 can be rotated mechanically so that the alignment bearings 251, 252, 253, 254 serve as the rotational coupling, or a separate rotational coupling can be provided.

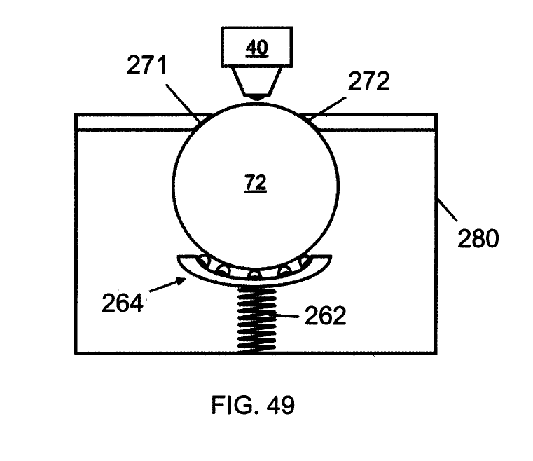

With reference to FIG. 49, the bearings can be other than roller bearings. For example, the bearings can be rollers, ball bearings, or bushing surfaces. In the variant test tube holder shown here, a housing 280 provides an anchor for a spring 262 that presses a set of biasing ball bearings 264 against the test tube 72 to press the test tube 72 against alignment bearings 271, 272 defined by bushing surfaces of the housing 280. Other types of bearings can be used for the biasing and/or alignment bearings that support the test tube as it rotates.

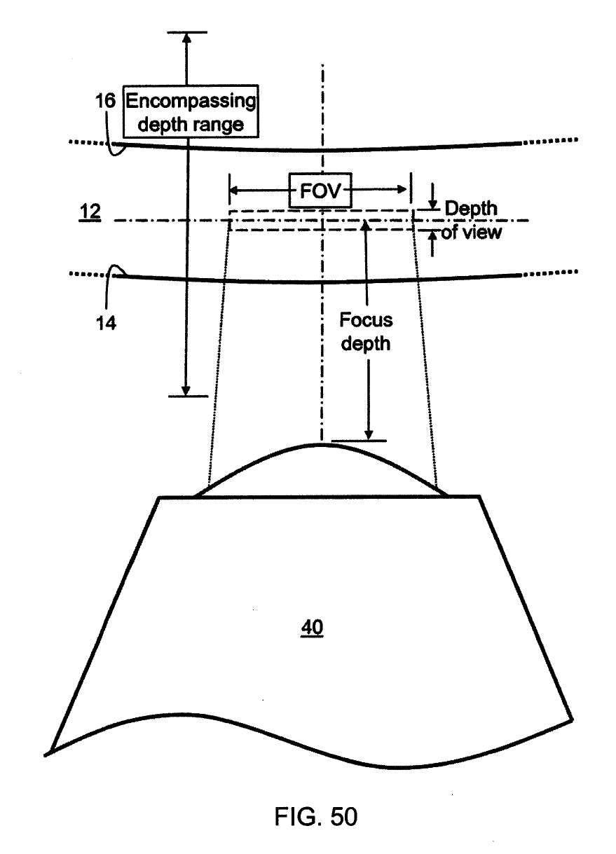

Suitable processing approaches for identifying or quantifying fluorescent dye tagged cells in an annular biological fluid layer are now described in FIGS. 50-56.

With reference to FIG. 50, certain measurement parameters are diagrammatically illustrated. The objective 40 images over a field of view (FOV) and over a depth of view located at a focus depth. Here, the focus depth is indicated respective to the objective 40; however, the focus depth can be denoted respective to another reference. In some embodiments, the depth of view of the objective 40 is about 20 microns, while the annular gap 12 between the test tube wall 14 and the float wall 16 is about 50 microns. However, the depth of focus corresponding to the annular gap 12 can vary substantially due to non-uniformities in the test tube and/or the float or other factors. It is expected that the annular gap 12 is located somewhere within an encompassing depth range. In some embodiments, an encompassing depth range of 300 microns has been found to be suitable. These dimensions are examples, and may be substantially different for specifice embodiments depending upon the specific objective 40, light-transmissive test tube, float, the type of centrifuging or other sample processing applied, and so forth.

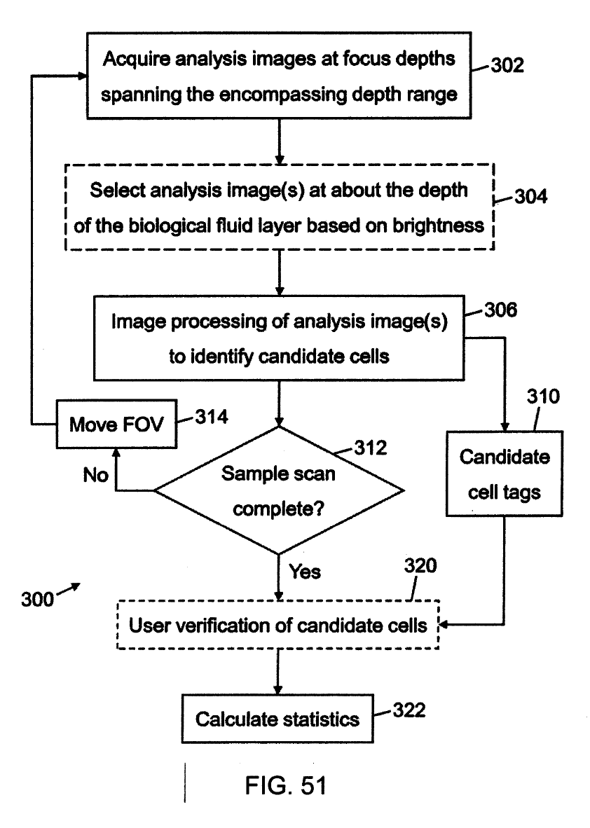

With reference to FIG. 51, one suitable data acquisition approach 300 is diagrammatically shown. In process operation 302, analysis images are acquired at a plurality of focus depths spanning the encompassing depth range. To avoid gaps in the depth direction, the number of analysis images acquired in the operation 302 should correspond to at least the encompassing depth range divided by the depth of view of the objective 40.

In some embodiments, the analysis images are processed in optional operation 304 to identify one or more analysis images at about the depth of the biological fluid layer (such as the buffy layer) based on image brightness. This optional selection takes advantage of the observation that typically the fluorescent dye produces a background fluorescence that is detected in the acquired analysis images as an increased overall image brightness. Image brightness can be estimated in various ways, such as an average pixel intensity, a root-mean-square pixel intensity, or so forth.

In an image processing operation 306, the analysis images, or those one or more analysis images selected in the optional selection operation 304, are processed using suitable techniques such as filtering, thresholding, or so forth, to identify observed features as candidate cells. The density of dye-tagged cells in the biological fluid layer is typically less than about one dye-tagged cell per field of view. Accordingly, the rate of identified candidate cells is typically low. When a candidate cell is identified by the image processing 306, a suitable candidate cell tag is added to a set of candidate cell tags 310. For example, a candidate cell tag may identify the image based on a suitable indexing system and x- and y-coordinates of the candidate cell feature. Although the density of rare cells is typically low, it is contemplated that the image processing 306 may nonetheless on occasion identify two or more candidate cells in a single analysis image. On the other hand, in some analysis images, no candidate cells may be identified.

At a decision point 312, it is determined whether the sample scan is complete. If not, then the field of view is moved in operation 314. For example, the field of view can be relatively scanned across the biological fluid sample in the annular gap 12 by a combination of rotation of the test tube 72 and translation of the objective 40 along the test tube axis 75. Alternatively, using the tube holder of FIG. 47, scanning is performed by moving the test tube 72 spirally. For each new field of view, the process operations 302, 304, 306 are repeated.

Once the decision point 312 indicates that the sample scan is complete, a user verification process 320 is optionally employed to enable a human analyst to confirm or reject each cell candidacy. If the image processing 306 is sufficiently accurate, the user verification process 320 is optionally omitted.

A statistical analysis 322 is performed to calculate suitable statistics of the cells confirmed by the human analyst. For example, if the volume or mass of the biological fluid sample is known, then a density of rare cells per unit volume or per unit weight (e.g., cells/milliliter or cells/gram) can be computed. In another statistical analysis approach, the number of confirmed cells is totaled. This is a suitable metric when a standard buffy sample configuration is employed, such as a standard test tube, standard float, standard whole blood sample quantity, and standardized centrifuging processing. The statistical analysis 322 may also include threshold alarming. For example, if the cell number or density metric is greater than a first threshold, this may indicate a heightened possibility of cancer calling for further clinical investigation, while if the cell number or density exceeds a second, higher threshold this may indicate a high probability of the cancer calling for immediate remedial medical attention.

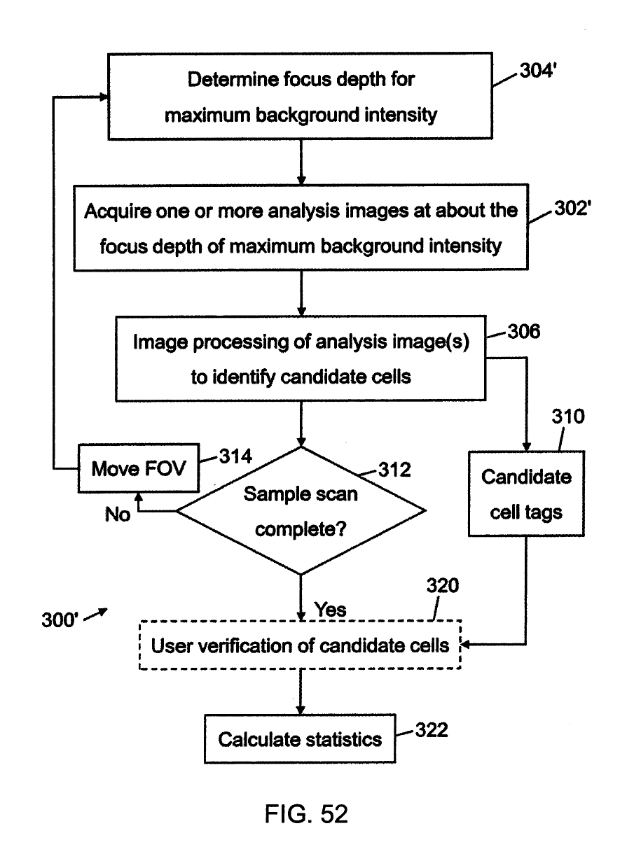

With reference to FIG. 52, a modified acquisition approach 300′ is diagrammatically shown. In modified process operation 304′, the focus depth for maximum background fluorescence intensity is first determined using input other than analysis images, followed by acquisition 302′ of one or a few analysis images at about the focus depth for maximum background fluorescence. For example, the search process 304′ can be performed by acquiring low resolution images at various depths. To avoid gaps in the depth direction, the number of low resolution images acquired in the operation 304′ should correspond to at least the encompassing depth range divided by the depth of view of the objective 40. In another approach, a large-area brightness sensor (not shown) may be coupled to the captured fluorescence 50 (for example, using a partial mirror in the camera 56, or using an intensity meter built into the camera 56) and the focus of the objective 40 swept across the encompassing depth range. The peak signal of the sensor or meter during the sweep indicates the focus providing highest brightness.

With the depth of the biological fluid sample determined by the process operation 304′, the acquisition process 302′ acquires only one or a few analysis images at about the identified focus depth of highest brightness. To ensure full coverage of the biological fluid layer, the number of acquired analysis images should be at least the thickness of the annular gap 12 divided by the depth of view of the objective 40. For example, if the annular gap 12 has a thickness of about 50 microns and the depth of view is about 20 microns, then three analysis images are suitably acquired—one at the focus depth of highest brightness, one at a focus depth that is larger by about 15-25 microns, and one at a focus depth that is smaller by about 15-25 microns.

An advantage of the modified acquisition approach 300′ is that the number of acquired high resolution analysis images is reduced, since the focus depth is determined prior to acquiring the analysis images. It is advantageous to bracket the determined focus depth by acquiring analysis images at the determined focus depth and at slightly larger and slightly smaller focus depths. This approach accounts for the possibility that the rare cell may be best imaged at a depth that deviates from the depth at which the luminescence background is largest.

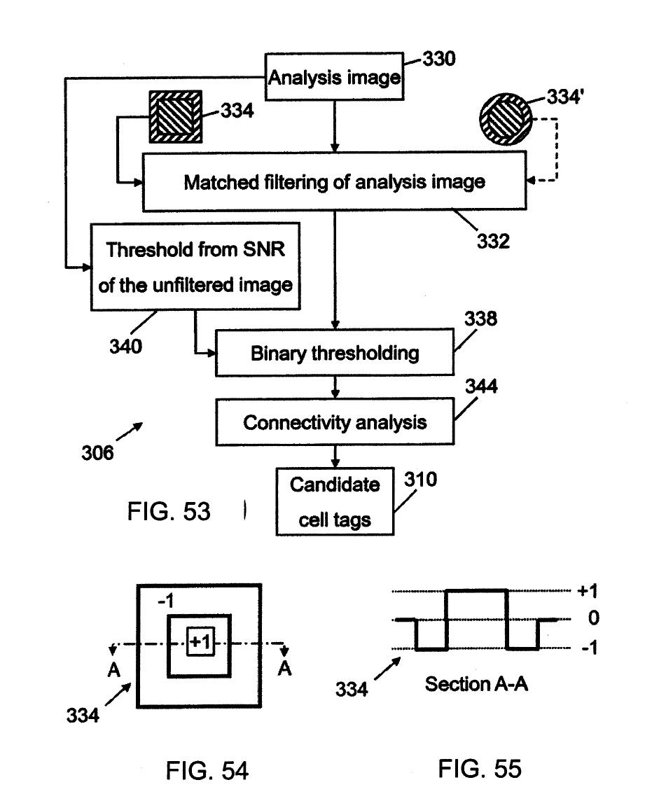

With reference to FIG. 53, a suitable embodiment of the image processing 306 is described, which takes advantage of a priori knowledge of the expected rare cell size to identify any cell candidates in an analysis image 330. In a matched filtering process 332, a suitable filter kernel is convolved with the image. The matched filtering 332 employs a filter kernel having a size comparable with the expected size of an image of a rare cell in the analysis image 330.

With continuing reference to FIG. 53 and with brief further reference to FIG. 54 and FIG. 55, in some embodiments a square filter kernel 334 is employed. The kernel 334 includes a central positive region of pixels each having a value of +1, and an outer negative region of pixels each having a value of −1. The area of the positive region should be about the same size as the area of the negative region. Points outside of either the inner or outer region have pixel values of zero. Optionally, other pixel values besides +1 and −1 can be used for the inner and outer regions, respectively, so as to give the filter a slightly positive or slightly negative response.

With continuing reference to FIG. 53, the matched filtering removes or reduces offsets caused by background illumination, and also improves the signal-to-noise ratio (SNR) for rare cells. The signal is increased by the number of points in the positive match area, while the noise is increased by the number of points in both the positive and negative match areas. The gain in SNR comes from the fact that the signal directly adds, while the noise adds as the root-mean-square (RMS) value or as the square root of the number of samples combined. For a filter with N positive points and N negative points, a gain of N/√(2N) or √(N/2) is obtained.

The square filter kernel 334 is computationally advantageous since its edges align with the x- and y-coordinate directions of the analysis image 330. A round filter kernel 334′ or otherwise-shaped kernel is optionally used in place of the square filter kernel 334. However, the round filter kernel 334′ is more computationally expensive than the square filter kernel 334. Another advantage of the square filter kernel 334 compared with the round filter kernel 334′ is that the total filter edge length of the square filter 334 is reduced from twice the detection size to 1.414 times the detection size. This reduces edge effects, allowing use of data that is closer to the edge of the analysis image 330.

The size of the filter kernel should be selected to substantially match the expected image size of a dye-tagged cell in the analysis image 330 to provide the best SNR improvement. For example, the square filter kernel 334 with a positive (+1) region that is ten pixels across is expected to provide the best SNR improvement for a cell image also having a diameter of about ten pixels. For that matched case, the signal is expected to increase by about a factor of 78 while the noise is expected to increase by about a factor of 14, providing a SNR improvement of about 5.57:1. On the other hand, the SNR improvement for a smaller eight pixel diameter cell using the same square filter is expected to be about 3.59:1. The SNR improvement for a larger fourteen pixel diameter cell using the same square filter is expected to be about 3.29:1.

The matched filter processing 332 can be implemented in various ways. In one approach, each point in the input image is summed into all points in the output image that are in the positive inner region. Then all the points in the output image that are in the outer negative region but not in the inner positive region are subtracted off. Each point in the input image is touched once, while each point in the output image is touched the outer-box pixel area count number of times.

In another suitable approach, for each point in the output image, all points from the input image that are within the positive inner box are read and summed. All points outside the positive inner box but within the negative outer box are then subtracted. While each output image pixel is touched only once, each input image pixel is touched by the outer-box pixel count.

In another suitable approach, two internal values are developed for the current row of the input image: a sum of all points in the row in the negative outer box distance, and a sum of all points in the row in the inner positive box distance. All output image column points at the current row have the input image sum of all points in the outer-box subtracted from them. All the output image column points within the inner positive box get the sum of the input image row points in the inner positive box distance added in twice. The row sums can be updated for the next point in the row by one add and one subtract. This reduces the execution cost to be on the order of the height of the filter box.

In the matched filter processing 332, various edge conditions can be employed. For example, in one approach, no output is produced for any point whose filter overlaps an edge of the analysis image 330. This approach avoids edge artifacts, but produces an output image of reduced usable area. In another suitable example edge condition, a default value (such as zero, or a computed mean level) is used for all points off the edge.

With continuing reference to FIG. 53, binary thresholding processing 338 is applied after the matched filtering 332. A difficulty in performing the thresholding 338 is selection of a suitable threshold value. Threshold selection is complicated by a likelihood that some analysis images will contain no cells, or only a single cell, or only a couple or few cells. In one approach, a the threshold is selected as a value that is a selected percentage below the peak pixel intensity seen in the filtered data. However, this threshold will cause noise to be detected when no cells are present, since in that case the peak pixel value will be in the noise. Another approach is to use a fixed threshold. However, a fixed threshold may be far from optimal if the background intensity varies substantially between analysis images, or if the matched filtering substantially changes the dynamic range of the pixel intensities.

In the illustrated approach, the threshold is determined by processing 340 based on the SNR of the unfiltered analysis image 330. By first determining the standard deviation of the input image, the expected noise at the filter output can be computed. The noise typically rises by the square root of the number of pixels summed, which is the outer-box area in pixel counts. In some embodiments, the threshold is set at approximately 7-sigma of this noise level. As this filter does not have an exact zero DC response, an appropriate mean level is also suitably summed to the threshold.

The thresholding 338 produces a binary image in which pixels that are part of a cell image generally have a first binary value (e.g., “1”) while pixels that are not part of a cell image generally have second binary value (e.g., “0”). Accordingly, connectivity processing 344 is performed to identify a connected group of pixels of the first binary value corresponding to a cell. The connectivity analysis 344 aggregates or associates all first binary value pixels of a connected group as a cell candidate to be examined as a unit. The center of this connected group or unit can be determined and used as the cell location coordinates in the candidate cell tag.

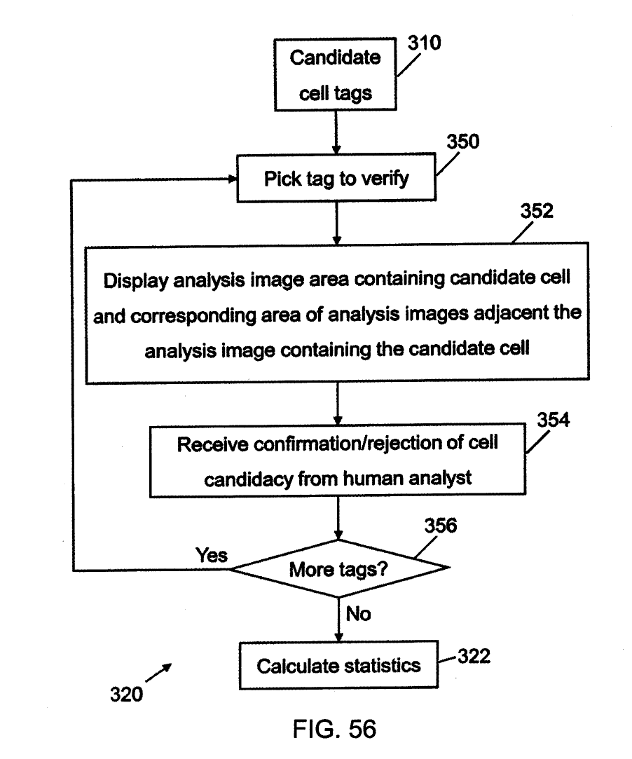

With reference to FIG. 56, a suitable embodiment of the optional user verification processing 320 is described. A tag is selected for verification in a selection operation 350. In a display operation 352, the area of the analysis image containing the candidate cell tag is displayed, optionally along with the corresponding area of analysis images adjacent in depth to the analysis image containing the candidate cell. Displaying the analysis images that are adjacent in depth provides the reviewing human analyst with additional views which may fortuitously include a more recognizable cell image than the analysis image in which the automated processing 306 detected the cell candidate. The human analyst either confirms or rejects the candidacy in operation 354. A loop operation 356 works though all the candidate cell tags to provide review by the human analyst of each candidate cell. The statistical analysis 322 operates on those cell candidate tags that were confirmed by the human analyst.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A method of testing for drug susceptibility in a cancer patient, comprising:
obtaining a control test tube (2320) and an assay test tube (2330), each test tube containing blood from the cancer patient;
adding a drug to the assay test tube (2340);
introducing a separator float into the assay test tube (2350);
moving the float into alignment with the cancer cells to capture the cancer cells in an annular volume (2360);
visually examining the assay test tube (2370); and
comparing the effect of the drug on cancer cells in the assay test tube to cancer cells in the control test tube (2380).

2. The method of claim 1, wherein the drug is a fluorescently labeled drug.

3. The method of claim 1, wherein a change in the shape of the cancer cells is compared between the assay test tube and the control test tube.

4. The method of claim 1, further comprising staining the cancer cells (2362) prior to visually examining the assay test tube.

5. The method of claim 1, wherein the visual examination is performed by detecting a quantity of fluorescence in the assay test tube.

6. The method of claim 1, further comprising visually examining the control test tube (2372).

7. The method of claim 1, wherein the movement of the float is performed by:
centrifuging the assay test tube to move the float into alignment with the cancer cells; and
reducing rotational speed to capture the cancer cells within an annular volume.

8. The method of claim 1, wherein the visual examination is performed using an optical system that generates light having a non-uniform spatial distribution.

9. The method of claim 1, wherein the separator float (1210) comprises a main body portion (1212), a plurality of axially oriented ridges (1224) protruding from the main body portion, and does not have end sealing ridges (1214).

10. The method of claim 1, wherein the control test tube and the assay test tube are obtained by receiving a blood sample (2310) from the cancer patient and dividing the blood sample (2315) into the control test tube (2320) and the assay test tube (2330).

11. The method of claim 1, wherein the control test tube and the assay test tube are obtained by receiving two test tubes (2320, 2330), each tube containing the blood of the cancer patient, wherein one test tube is designated as the control test tube and the other test tube is designated as the assay test tube.

12. The method of claim 1, wherein a plurality of assay test tubes are obtained;
wherein the drug is added to each assay test tube, wherein the amount of the drug is different in each assay test tube; and
comparing the effect of the amount of the drug on cancer cells in each assay test tube with cancer cells in the control test tube.

13. The method of claim 1, wherein the drug is added to the assay test tube (2340) after moving the float into alignment with the cancer cells to capture the cancer cells in an annular volume (2360).

14. The method of claim 1, wherein the drug is added to the assay test tube (2340) before moving the float into alignment with the cancer cells to capture the cancer cells in an annular volume (2360)

15. A method of testing for drug susceptibility in a cancer patient, comprising:
receiving a first test tube and a second test tube, each tube containing the blood of the cancer patient;
adding a drug (2340) to the first test tube to make an assay test tube (2330);
visually examining the assay test tube (2370); and
comparing the effect of the drug on cancer cells in the assay test tube with cancer cells in the control test tube (2380).

16. A method of quantifying the appropriate dose of a drug for a patient, comprising:
dividing a blood sample (2315) of the cancer patient into a control test tube (2320) and a plurality of assay test tubes (2330);
adding a drug to each assay test tube, wherein the amount of the drug is different in each assay test tube (2340);
introducing a separator float into each assay test tube (2350);
moving the float into alignment with the cancer cells to capture the cancer cells in an annular volume in each assay test tube (2360);
visually examining each assay test tube (2370); and
comparing the effect of the amount of the drug on cancer cells in each assay test tube with cancer cells in the control test tube (2380).

17. The method of claim 16, wherein a change in the shape of the cancer cells or the number of intact cancer cells is compared between the assay test tubes.

18. The method of claim 16, further comprising staining the cancer cells (2362) prior to visually examining the assay test tubes.

19. The method of claim 16, wherein the drug is a fluorescently labeled drug.
